(19) United States
(12) Patent Application Publication
(10) Pub. No.: US 2006/0251720 A1
(43) Pub. Date: Nov. 9, 2006

(54) Localized controlled absorption of statins in the gastrointestinal tract for achieving high blood levels of statins

(76) Inventors: Adel Penhasi, Holon (IL); Maxim Gomberg, Jerusalem (IL)

Correspondence Address: WINSTON & STRAWN LLP, 1700 K STREET, N.W., WASHINGTON, DC, 20006, US

(21) Appl. No.: 11/305,544

(22) Filed: Dec. 15, 2005

Related U.S. Application Data
(63) Continuation of application No. PCT/IL05/00539, filed on May 26, 2005.
(60) Provisional application No. 60/574,561, filed on May 27, 2004.
(60) Provisional application No. 60/590,919, filed on Jul. 26, 2004.

(51) Int. Cl. A61K 31/401; A61K 31/366; A61K 31/22; A61K 9/22

(52) U.S. Cl. 424/468; 514/423; 514/460; 514/548

(57) ABSTRACT

The present invention relates to a localized controlled absorption formulation of a statin in which rapid release of the active ingredient preferentially occurs in the lower gastrointestinal tract including the colon. The formulation provides significantly higher blood level concentration and bioavailability of the active ingredient in the body of a subject as compared to the bioavailability achieved from the currently available conventional formulations The blood levels are maintained for a significantly longer period of time as compared with currently available conventional formulations. The formulation preferably includes a core, over which an outer coating is layered. The core preferably includes a burst controlling agent and optionally a disintegrant. The outer coating includes a water insoluble polymer and at least one water permeable agent allowing entry of water into said core, the water permeable agent comprising hydrophilic particulate matter. The core is preferably in the form of a tablet.

### CROSS-REFERENCE TO RELATED APPLICATIONS

This application is a continuation of International application PCT/IL2005/000539 filed May 26, 2005, and claims the benefit of provisional application 60/574,561 filed May 27, 2004 and 60/590,919 filed Jul. 26, 2004: the entire content of each of which is expressly incorporated herein by reference thereto.

### FIELD OF THE INVENTION

The present invention relates to a formulation for the localized controlled absorption of a medication, and in particular, to a formulation for the delayed onset, rapid burst release of HMG-CoA reductase inhibitors (statins), predominantly in the lower gastrointestinal (GI) tract for achieving high blood levels of statins.

### BACKGROUND OF THE INVENTION

Controlled release formulations for oral administration of drugs are beneficial for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may decrease, leading to a more even drug level in the blood over a period of time. Such formulations may also provide a longer plateau concentration of the drug in the blood.

Currently, sustained and controlled release drug delivery systems administered by the oral route are usually based on either a gel forming matrix or are coated formulations, or a combination thereof.

A drug delivery system should preferentially deliver drugs to any part of the lower gastrointestinal (GI) tract, as a site for topical delivery and subsequent absorption of the drug. This concept relies on the fact that the retention time of the drug delivery system through the colon may be the longest as compared to other parts of gastrointestinal tract. Likewise, such a delivery system can also advantageously use the unique continuous absorption characterizing the colon, which results in more flat, consistent concentration levels of the drug in blood. Such absorption, of course, can contribute significantly to reduction of the fluctuations in blood drug concentration, thus preventing the side effects which may appear upon using either immediate or conventional controlled release formulations, thereby improving compliance.

Many different types of controlled release formulations for delivery to the colon are known in the art. These include pH-dependent delivery systems and time-dependent delivery systems. Time-dependent systems release the drug load after a pre-programmed time delay. To attain colonic release, the lag time should equal the time taken for the drug delivery system to reach the colon. The small intestinal transit time is generally considered to be in the region of three to four hours.

The statins are a class of compounds which contain a moiety that can exist as either a 3-hydroxy lactone ring or as the corresponding open ring dihydroxy acid. The structural formulas of these and additional HMG-CoA reductase inhibitors are described, inter alia, in M. Yalpani, “Cholesterol Lowering Drugs”, Chemistry & Industry, pp. 85-89 (1996).

The statins are orally effective in the reduction of serum cholesterol levels, by competitively inhibiting 3-hydroxy-3-methylglutaryl coenzyme A (HMG CoA) reductase, and play an important role in primary and secondary prevention of ischemic heart disease and myocardial infarction.

The statins include natural fermentation products lovastatin (described in U.S. Pat. No. 4,231,938) and mevastatin (described in U.S. Pat. No. 3,671,523), as well as a variety of semi-synthetic and totally synthetic products, which include simvastatin (U.S. Pat. No. 4,444,784); pravastatin sodium salt (U.S. Pat. No. 4,346,227); fluvastatin sodium salt (U.S. Pat. No. 5,354,772); atorvastatin calcium salt (U.S. Pat. No. 5,273,995); cerivastatin sodium salt (also known as rivastatin; U.S. Pat. No. 5,177,080) and pitavastatin (U.S. Pat. No. 5,854,259, U.S. Pat. No. 5,856,336, U.S. Pat. No. 5,872,130, U.S. Pat. No. 5,011,930).

An osmosis-controlled release formulation for a statin is taught in U.S. Pat. No. 5,916,595 to Andrx, which comprises a core containing a water swellable polymer, an osmotic agent; and an outer coating which comprises a pH sensitive coating agent, a channeling agent and a water insoluble cellulose polymer. Water is drawn into the tablet, which expands to the point where the outer coating fails in one particular area to form a constricted opening which releases the internal contents of the tablet which contain the drug. Thereafter, the aqueous medium of the tablet shell continues to release the drug as it dissolves until the osmotic pressure inside the tablet shell equals that of the surrounding environment. At the late stages of the in vivo release, the tablet shell collapses and/or disintegrates completely in order to substantially release the remaining drug. Complete release occurs over a period of 4-30 hours.

U.S. Pat. No. 5,882,682 to Merck teaches controlled delivery of simvastatin from a core by use of a water insoluble coating which contains apertures. The release rate of the simvastatin is a function of the number and size of the apertures in the coating, and is a slow, extended form of release.

U.S. Pat. No. 4,997,658 to Merck teaches a method for lowering plasma cholesterol by using a HMG-CoA reductase inhibitor in a sustained release manner over a period of 6-24 hours, as a slow, extended form of release.

WO 01/34123 to Andrx teaches a controlled release dosage form of a drug which may include statins, in which the release is gradual, and occurs at about 10 to about 32 hours after oral administration; the drug emerges from the formulation in a slow, extended form of release.

WO 2004/021972 to Biovail purports to provide methods to release a statin in the lower gastrointestinal tract thereby decreasing metabolism of the statin prior to its absorption. The proposed formulations disclosed in that application putatively decrease the concentration of lovastatin and simvastatin and their active metabolites in the systemic circulation and at the same time provide increased concentrations of these statins in the liver. The disclosure teaches extended release formulations as preferred over a burst release formulation, and the structure of the formulations taught may for example feature a number of compartments.

A gastrointestinal controlled delivery system is disclosed in U.S. Pat. Nos. 5,840,332 and 6,703,044, neither of which relate to the use of those formulations for very poorly water soluble drugs in general and make no reference whatsoever to statins in particular.

Various references teach the metabolism and pharmacokinetics of statins in the human body (see for example M. J. Garcia et al., Clinical Pharmacokinetics of Statins, Clin. Pharmacol. 2003, 25 (6): 457-481).

Simvastatin is administered as the inactive lactone prodrug that must be hydrolyzed in the plasma and liver to the beta-hydroxy acid form for pharmacological activity. Simvastatin is believed to be metabolized in the liver and intestine, at least by the enzyme CYP3A, considering the beta-hydroxy acid form as the drug, the major active metabolites are 6-beta-hydroxymethyl and 6-beta-hydroxy simvastatin, which retain approximately 40% and 50%, respectively, of HMG-CoA reductase activity. Absorption reaches 60% while the bioavailability of the beta-hydroxy acid form following oral administration of simvastatin is less than 5%.

The poor bioavailability of simvastatin is mainly attributed to its low solubility in gastrointestinal fluids, low permeability through the mucosal membrane, and extensive first-pass metabolism. Since simvastatin (as stated above) is believed to be a CYP3A4 substrate, simvastatin may be expected to undergo significant intestinal metabolism.

The above cited reference also teaches that about 87% of the absorbed dose of simvastatin undergoes hepatic metabolism. The activation of simvastatin is by carboxyesterase-mediated hydrolysis, which occurs to a slight extent in plasma and to a higher extent in the liver. Both the parent lactone and the acid forms are normally present in very small amounts in the plasma, due to a high hepatic extraction ratio.

Simvastatin and its active acid forms are highly bound to plasma proteins, primarily to albumin (more than 95%). More than 98% of simvastatin is protein bound versus 94.5% for the open hydroxy acid form. As only unbound drug is assumed to be able to enter the tissues, the high protein binding and low plasma concentrations of simvastatin are in agreement with the low peripheral tissue exposure in humans.

Physicians' Desk Reference 58th edition, 2004, pages 2113-2118 teaches the metabolism, pharmacokinetics, pharmacodynamics and side effects of simvastatin, and is hereby incorporated by reference as if fully set forth herein.

The background art does not teach or suggest a delayed onset rapid burst release formulation for delivery of statins to the lower GI tract including the colon. Nor does the art teach or suggest a delayed onset rapid burst formulation, which provides greater bioavailability and higher blood concentrations of the statins. The background art also does not teach or suggest such a formulation, which provides fewer side effects, for greater patient compliance and comfort.

There remains an unmet need for formulations of statins with improved bioavailability and pharmacokinetics of the active species which provide high blood levels of statins, while minimizing side effects.

### SUMMARY OF THE INVENTION

The present invention overcomes the deficiencies of known formulations of statins by providing a localized controlled absorption formulation, preferably for once a day administration, in which rapid release of the active ingredient preferably occurs in the lower GI tract including the colon. Alternatively, such release can occur in the small intestine. The formulation provides significant plasma levels of a statin, its pharmaceutically acceptable salts and esters, or its metabolites that are maintained for an extended period after administration, preferably for at least about 12 hours and more preferably for at least about 24 hours after the burst release occurs.

Without wishing to be limited by a single hypothesis or theory, the formulations of the present invention are believed to have preferential release of the drug in the lower GI tract, resulting in increased amount of a statin and its active hydroxy acid forms than would have been formed if the drug were allowed to be absorbed into the bloodstream prior to reaching the appropriate section(s) of the intestine.

Local intestinal production of a greater amount of the active metabolite, probably through the activity of colonic natural flora, or via other metabolic routes, will further enhance the desired clinical effect and allow the achievement of intestinal drug levels of these metabolites that are unattainable by systemic or conventional oral delivery.

By using the formulation according to the present invention which is preferably a delayed burst fast release (hereinafter: DBR) formulation, it may be possible to obtain increased production of active forms in the gut than that which can be obtained through carboxyesterse-mediated hydrolysis in the liver.

Further advantages of at least partial colonic delivery are that statins probably have greater solubility in the colon, and colon transit times are longer, resulting in increased time of exposure of the drug, and hence greater absorption.

Orally administered drugs or chemical agents that are processed to active forms in the intestinal environment can be administered to a patient who suffers from impaired liver function. Impaired liver function prevents or diminishes the normal hepatic metabolism of drugs to active metabolites. The increased production of active forms following administration of the formulations of the present invention is believed to reduce stress on the liver. The liver enzyme CYP3A4 is also present in the intestine, hence metabolism in the intestine can serve an alternative for metabolism in the liver for such drugs in these patients.

One of the main advantages of the present invention is that a reduced dosage of a statin can be used in comparison to presently available formulations, which can lead to the following beneficial effects:

1. Reduced liver side effects, such as a reduced level of transaminase for example (dose-related side effect).

2. Reduced incidence of rhabdomyolysis, muscle pain, and/or reduced level of CPK (dose-related side effect).

3. Reduced gastrointestinal effects including but not limited to nausea, dyspepsia, flatulence, and/or constipation (may be dose related side effects).

4. Better tolerated multiple drug treatment in which at least one additional drug is metabolized by the liver.

5. Improved patient compliance.

A further advantage of the present invention is that a reduced food effect on the release may be obtained, since the formulation according to the present invention releases the active ingredient predominantly in the lower gastrointestinal tract including the colon. Metabolism and absorption of orally administered drugs are commonly known to be affected by interactions with food. The formulation of the present invention is expected to be only slightly affected or even unaffected by such interactions, since metabolism and absorption of the statin occurs in the intestine, optionally and preferably in the colon.

According to a first aspect, the formulation according to the present invention is a drug delivery formulation, preferably a delayed burst release formulation, for localized drug release of a statin in the gastrointestinal tract comprising a core, over which an outer coating is layered. The core comprises at least one statin or a pharmaceutically acceptable salt or ester thereof, and at least one burst controlling agent; and the outer coating comprises a water insoluble hydrophobic carrier and a water insoluble hydrophilic particular matter. The hydrophilic particular matter is preferably a water permeable agent which allows entry of liquid into the core.

In one preferred embodiment, the coating surrounding the drug containing core comprises a water-insoluble hydrophilic particulate matter embedded in the hydrophobic water-insoluble carrier, such that when the formulation enters the gastrointestinal tract, the particulate matter absorbs liquid, thus forming channels that interconnect the core with the outer surface of the coating, and through which channels, liquid reaches the burst controlling agent in the core. According to one embodiment, the drug from the core is released into the gastrointestinal tract through these channels.

In one embodiment, the formulations of the present invention are characterized in that the in vivo blood plasma concentration of the statin or a pharmaceutically acceptable salt or ester thereof in the subject is substantially zero for at least about 1.5 hours after oral administration of the formulation. In one embodiment, the formulations of the present invention are characterized in that the in vivo blood plasma concentration of the statin or a pharmaceutically acceptable salt or ester thereof in the subject is substantially zero for at least about 2 hours after oral administration of the formulation. In another embodiment, the in vivo blood plasma concentration of the statin or a pharmaceutically acceptable salt or ester thereof in the subject is substantially zero for at least about three hours after oral administration of the formulation. In yet another embodiment, the in vivo blood plasma concentration of the statin or a pharmaceutically acceptable salt or ester thereof in the subject is substantially zero for at least about four hours after oral administration of the formulation

According to one embodiment, the delayed burst release formulation of the present invention provides an increased amount of a statin, a pharmaceutically acceptable salt or ester thereof, or an active form thereof to the circulation of a subject, compared to a substantially similar dose of a conventional immediate release formulation of the stain.

According to an alternative embodiment, the delayed burst release formulation of the present invention provides enhanced bioavailability of a statin, a pharmaceutically acceptable salt or ester thereof, or an active form thereof in a subject, compared to a substantially similar dose of an immediate release formulation of the statin.

According to yet another alternative embodiment, the delayed burst release formulation of the present invention provides a therapeutically effective amount of a statin, a pharmaceutically acceptable salt or ester thereof, or an active form thereof into the circulation of a subject.

According to yet another alternative embodiment, the delayed burst release formulation of the present invention provides a therapeutically effective amount of a hydroxy acid metabolite of a statin or a pharmaceutically acceptable salt or ester thereof into the circulation of a subject.

As contemplated herein, one of the advantages of the novel delayed burst release formulations of the present invention, is that they preferentially release the statin in the gastrointestinal tract of the subject as opposed to conventional immediate release formulations and certain controlled release formulations. Such localized delivery leads to increased efficacy and accordingly, the formulations of the present invention are believed to be capable of providing at least a similar, if not greater, pharmaceutical effect of the active ingredient using a significantly lower dosage amounts as compared to other orally administered formulations known in the art. Thus, according to one embodiment, the statin is present in the formulations of the present invention in a decreased dosage amount of up to about 60% as compared to an immediate release formulation of the same statin, while providing a substantially similar therapeutic effect to the immediate release formulation.

Another significant advantage of the delayed release formulations of the present invention, is that they achieve in-vivo blood levels of the statins or their active metabolites, for an extended period of time, as compared with conventional immediate release statin formulations. In one embodiment, the formulations of the present invention are characterized in that they provide therapeutically effective amounts of the statin, its pharmaceutically acceptable salt or ester thereof, or its active form in the subject for at least about 12 hours after the burst release occurs, preferably for at least about 24 hours after the burst release occurs.

The delayed burst release formulations preferably release substantially no statin in vitro preferably for at least about 1 hour, or for at least about 1.5 hours, or for at least about 2 hours. Preferably, at least about 70% of the statin is released in vitro about one hour after the delayed burst release occurs.

Preferably the formulation releases the statin in the lower GI, in the cecum, and/or in the colon of the subject.

In another embodiment, the present invention provides a method for using a delayed burst release formulation according to the present invention, for providing treatment for high blood cholesterol to a subject in need thereof.

In a further aspect, the present invention relates to a method for providing a therapeutically effective amount of a statin, a pharmaceutically acceptable salt or ester thereof or an active form thereof to a subject, comprising orally administering to the subject a delayed burst release formulation comprising (a) a core comprising at least one statin or a pharmaceutically acceptable salt or ester thereof. and at least one burst controlling agent; and (b) an outer coating over the core, the outer coating comprising a water insoluble hydrophobic carrier and a water insoluble hydrophilic particular matter, the water insoluble hydrophilic particulate matter allowing entry of liquid into the core.

In yet a further aspect, the present invention relates to a method for providing enhanced bioavailability of a statin, a pharmaceutically acceptable salt or ester thereof or an active form thereof to a subject, comprising orally administering to the subject a delayed burst release formulation comprising (a) a core comprising at least one statin or a pharmaceutically acceptable salt or ester thereof. and at least one burst controlling agent; and (b) an outer coating over the core, the outer coating comprising a water insoluble hydrophobic carrier and a water insoluble hydrophilic particular matter, the water insoluble hydrophilic particulate matter allowing entry of liquid into the core.

In yet a further aspect, the present invention provides a method of providing fast release of a statin a pharmaceutically acceptable salt or ester thereof or an active form thereof in the gastrointestinal tract of a subject, comprising orally administering to the subject a delayed burst release formulation comprising (a) a core comprising at least one statin or a pharmaceutically acceptable salt or ester thereof. and at least one burst controlling agent; and (b) an outer coating over the core, the outer coating comprising a water insoluble hydrophobic carrier and a water insoluble hydrophilic particular matter, the water insoluble hydrophilic particulate matter allowing entry of liquid into the core.

In yet a further aspect, the present invention provides a method for providing a therapeutically effective amount of a statin, a pharmaceutically acceptable salt or ester thereof or an active form thereof to a subject, comprising orally administering to the subject a delayed burst release formulation comprising a) a core comprising at least one statin or a pharmaceutically acceptable salt or ester thereof. and at least one burst controlling agent; and (b) an outer coating over the core, the outer coating comprising a water insoluble hydrophobic carrier and a water insoluble hydrophilic particular matter, the water insoluble hydrophilic particulate matter allowing entry of liquid into the core, characterized in that the formulation provides a therapeutically effective amount of said statin, a pharmaceutically acceptable salt or ester thereof or an active form thereof in the subject for at least about 12 hours, preferably for at least about 24 hours after the burst release occurs. In one embodiment, the formulation releases the statin in the gastrointestinal tract, and provides clinically effective amounts of said hydroxy acid metabolite of said statin into the circulation of the subject for at least about 12 hours, preferably for at least about 24 hours after the burst release occurs.

According to various alternative embodiments, the core of the formulations of the present invention is selected from the group consisting of a compressed tablet, a pellet, a pill, microparticles, an agglomerate, a capsule or any other solid dosage form known to a person of skill in the art. Preferably, the core is a tablet.

According to various alternative embodiments, the statin is selected from lovastatin, mevastatin simvastatin, pravastatin, fluvastatin, atorvastatin, and cerivastatin also known as rivastatin, pitavastatin and salts, esters and active forms thereof, as defined herein. The dosage levels of the active ingredient can easily be determined by one of ordinary skill in the art. According to certain currently preferred embodiments, the statin is selected from simvastatin, atorvastatin, pitavastatin and lovastatin.

The burst controlling agent in the core preferably comprises a water insoluble polymer for controlling the rate of penetration of water into the core and raising the internal pressure (osmotic pressure) inside the core. Such a burst controlling agent is preferably able to swell upon contact with liquid. According to various alternative embodiments, the water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

According to specific embodiments, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof. According to specific embodiments, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose. According to certain currently preferred embodiments, the water insoluble polymer is calcium pectinate, microcrystalline cellulose, or a combination thereof.

According to various alternative embodiments, the core further comprises at least one disintegrant. According to specific embodiments, the disintegrant is selected from the group consisting of croscarmellose sodium, crospovidone (cross-linked PVP) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. More preferably, the disintegrating agent is croscarmellose sodium.

According to various alternative embodiments, the core further comprises at least one of an absorption enhancer, a binder, a hardness enhancing agent, a buffering agent, a filler, a flow regulating agent, a lubricant, a synergistic agent, a chelator, an antioxidant, a stabilizer and a preservative, and optionally one or more other excipients.

According to various alternative embodiments of the present invention, the hydrophobic water insoluble carrier of the outer coating is selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes. More preferably, the water insoluble polymer is ethylcellulose.

According to certain embodiments, the water insoluble particulate matter in the outer coating is a hydrophilic yet water insoluble polymer, preferably selected from the group consisting of a water insoluble cross-linked polysaccharide, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross-linked cellulose derivatives, water insoluble cross-linked polyvinyl pyrrolidone, micro crystalline cellulose, insoluble starch, micro crystalline starch and a combination thereof. Most preferably, the water insoluble particulate matter is microcrystalline cellulose.

Optionally, the outer coating further comprises at least one of a wetting agent, a suspending agent, a dispersing agent, a stiffening agent and a plasticizer.

Optionally, the formulation comprises an enteric coating disposed on the outer coating. The enteric coating is preferably selected from the group consisting of cellulose acetate phthalate, hydroxy propyl methyl cellulose acetate succinate, poly(methacrylic acid, methyl methacrylate) 1:1 and (Eudragit L100), poly(methacrylic acid, ethyl acrylate) 1:1 (Eudragit L30D-55).

It is apparent to a person skilled in the art that the present invention is not limited to the particular delayed burst release formulations described herein, and that any delayed burst release formulation of statins, their pharmaceutically acceptable salts or esters or their active forms, that 1) provides an increased amount of the statin to the circulation of a subject; 2) provides enhanced bioavailability of the statin in a subject; 3) provides fast release of the statin in a subject; 4) includes a reduced amount of the statin (preferably up to 60%) as compared to an immediate release formulation of the statin, while providing a substantially similar therapeutic effect to the immediate release formulation; 5) releases substantially no statin in vitro for at least about 1 hour, preferably for at least about 1.5 hours, more preferably for at least about 2 hours; 6) releases at least about 70% of the statin in vitro about one hour after the delayed burst release occurs; 7) provides substantially zero in vivo blood plasma concentrations of the statin for at least about 1.5 hours or 2 hours after oral administration; and 8) provides therapeutically effective amounts of the statin at least about 12 hours, preferably for about 24 hours after the burst release occurs, is encompassed within the broad scope of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention is herein described, by way of example only, with reference to the accompanying drawings, wherein:

FIGS. 1 to 4 show in vitro dissolution profiles of statins from formulations containing 16 mg simvastatin (FIG. 1); 16 mg simvastatin and an enteric coating (FIG. 2); 40 mg simvastatin (FIG. 3); and 10 mg simvastatin (FIG. 4), prepared according to the present invention.

FIG. 5 shows the release profile of simvastatin from an uncoated core formulation.

FIGS. 6 to 9 show mean in vivo plasma concentration-time curves for the simvastatin (FIGS. 6 and 8) and beta-hydroxy-acid simvastatin (FIGS. 7 and 9) for a formulation of the present invention containing 16 mg and 40 mg simvastatin as compared to a reference formulation.

### DETAILED DESCRIPTION OF THE PREFERRED EMBODIMENTS

The present invention provides a formulation for the controlled delivery of a statin to the gastrointestinal tract. The formulation comprises a drug containing core surrounded by a coating that limits the access of liquid to the core thereby controlling the release of the drug from the core to the GI tract. The formulation provides controlled absorption of the statin, adapted so as to provide a time-delayed, burst fast release in the colon or small intestine. The formulation supports a lag time between oral administration and release of the active ingredient, providing higher bioavailability and lower dosage as compared to currently used formulations. The formulation of the present invention preferably includes non pH-dependent release.

Thus, in one embodiment, the present invention provides a delayed burst release oral formulation for localized release of a statin in the gastrointestinal tract of a subject, comprising: (a) a core comprising at least one statin or a pharmaceutically acceptable salt or ester thereof, and at least one burst controlling agent; and (b) an outer coating over the core, the outer coating comprising a water insoluble hydrophobic carrier and a water insoluble hydrophilic particular matter, the water insoluble hydrophilic particulate matter allowing entry of liquid into the core. The term “localized release”, as used herein, means providing a high local concentration of drug released from the delayed burst release (DBR) formulation in a specific site of the gastrointestinal tract.

In one embodiment, the in vivo blood plasma concentration of the statin and/or a pharmaceutically acceptable salt and/or ester thereof is controlled by a lag time, providing a controlled absorption of the statin and/or a pharmaceutically acceptable salt and/or ester thereof and/or related active forms. In one specific embodiment, the formulations of the present invention are characterized in that the in vivo blood plasma concentration of the statin or a pharmaceutically acceptable salt or ester thereof in the subject is substantially zero for at least about 1.5 hours after oral administration of the formulation. In another specific embodiment, the formulations of the present invention are characterized in that the in vivo blood plasma concentration of the statin or a pharmaceutically acceptable salt or ester thereof in the subject is substantially zero for at least about two hours after oral administration of the formulation. In another specific embodiment, the in vivo blood plasma concentration of the statin or a pharmaceutically acceptable salt or ester thereof in the subject is substantially zero for at least about three hours after oral administration of the formulation. In yet another specific embodiment, the in vivo blood plasma concentration of the statin or a pharmaceutically acceptable salt or ester thereof in the subject is substantially zero for at least about four hours after oral administration of the formulation. The term “substantially zero”, as used herein, means that the statin is either not detected in the blood, or only minor amounts of the statin are detected in the blood.

According to one embodiment, the delayed burst release formulation of the present invention provides an increased amount of a statin, a pharmaceutically acceptable salt or ester thereof, or an active form thereof to the circulation of a subject, compared to a substantially similar dose of a conventional immediate release formulation of the stain. As used herein, the term “substantially similar dose” means a dose which is either equivalent or is substantially similar, for example a difference of not more than about 25%. The term “increased amount” means that administration of the formulations of the present invention result in higher blood levels of the statins or their active metabolites (e.g., 10% higher, 20% higher, 50% higher 100% higher, 200% higher, 500% higher etc.), as compared with blood levels achieved by administration of conventional statin formulations. The levels of the statins can be measured by determining the plasma concentration of the statins as a function of time following administration of the formulation, as known to a person of skill in the art. As demonstrated herein, administration of several simvastatin and pitavastatin formulations according to the present invention to subjects resulted in blood levels that were significantly higher than the blood levels achieved after administration of conventional formulations of these statins. Further, importantly, the blood levels were maintained for significantly longer time periods as compared with the conventional formulation. For example, blood levels can be maintained for at least about 6 hours, preferably for about 8 hours, about 10 hours, about 12 hours and most preferably for about 24 hours after the delayed burst release occurs.

According to an alternative embodiment, the delayed burst release formulation of the present invention provides enhanced bioavailability of a statin, a pharmaceutically acceptable salt or ester thereof, or an active form thereof in a subject, compared to a substantially similar dose of an immediate release formulation of the stain. The term “enhanced bioavailability” means that administration of the formulations of the present invention results in higher bioavailability of the statins or their active metabolites (e.g., 10% higher, 20% higher, 50% higher 100% higher, 200% higher, 500% higher etc.), as compared with the bioavailability achieved by administration of conventional statin formulations. Bioavailability can be measured for example by comparing the AUC values obtained after administration of the formulations, as known to a person of skill in the art. As demonstrated herein, administration of several simvastatin and pitavastatin formulations according to the present invention to subjects resulted in AUC values that were more than two fold higher than the AUC values obtained after administration of conventional formulations of these statins. Further, the AUC values were maintained for significantly longer time periods as compared with the conventional formulation, for example for at least about 6 hours, preferably for about 8 hours, about 10 hours, about 12 hours and most preferably for about 24 hours after the delayed burst release occurs.

According to yet another alternative embodiment, the delayed burst release formulation of the present invention provides a therapeutically effective amount of a statin, a pharmaceutically acceptable salt or ester thereof, or an active form thereof into the circulation of a subject. The term “therapeutically effective amount” refers to an amount of the statin which will result in a therapeutic effect of the disease or condition being treated, for example high blood cholesterol.

The present invention represents an improvement over WO 2004/021972 to Biovail, as the Biovail application seeks to reduce the concentration of statins in the blood circulation. In contrast, the present invention provides an increased concentration of statins or active forms thereof in the blood circulation thus resulting in the administration of relatively lower dose of a statin or active forms thereof in the formulation administered to the subject (patient), thereby potentially reducing side effects by decreasing the total dose of statin to which the body of the subject is exposed.

As explained above, the statins are a class of compounds which contain a moiety that can exist as either a 3-hydroxy lactone ring or as the corresponding open ring dihydroxy acid. Typically, the statins can be administered as the inactive lactone prodrugs that must be hydrolyzed in the plasma and liver to the beta-hydroxy acid form for pharmacological activity. In accordance with the present invention, the delayed burst release formulations described herein are capable of providing a therapeutically effective amount of the hydroxy acid metabolite of a statin or a pharmaceutically acceptable salt or ester thereof into the circulation of a subject.

Further contemplated within the broad scope of the present invention is a delayed burst release pharmaceutical formulation comprising a core and an outer coating that surrounds the core; the core comprising a relatively lower dose of a statin, or pharmaceutically acceptable salts and/or esters thereof, and optionally at least one burst controlling agent and at least one disintegrant; and the outer coating comprising a water insoluble hydrophobic carrier and water-insoluble but hydrophilic particulate matter. By “relatively lower dose” it is meant a dose that provides at least the same or similar pharmaceutical and/or therapeutic effect (if not a greater effect) as a conventional dose of a statin, while featuring a lower amount of statin than the conventional dose of the statin. It should be noted that a similar principle may optionally be applied for dosage forms featuring a plurality of different statins.

In yet another embodiment, the present invention provides a formulation that provides a statin and/or a pharmaceutically acceptable salt and/or ester thereof for administration to a subject, comprising: a delayed burst release formulation for oral administration comprising a core and an outer coating that surrounds the core; the core comprising a statin and/or a pharmaceutically acceptable salt and/or ester thereof, and optionally at least one burst controlling agent and at least one disintegrant; and the coating comprising a water-insoluble hydrophobic carrier and a hydrophilic particulate matter

In yet another embodiment, the present invention provides a formulation that provides an increased amount of simvastatin and/or an active form of simvastatin and/or pharmaceutically acceptable salt and/or ester thereof in the blood of a subject, comprising a delayed burst release formulation for oral administration comprising a core and an outer coating that surrounds the core the core comprising simvastatin and/or a pharmaceutically acceptable salt and/or ester thereof, and optionally at least one burst controlling agent and at least one disintegrant; and the coating comprising a water-insoluble hydrophobic carrier and a hydrophilic particulate matter.

In yet another embodiment, the present invention provides a delayed burst release formulation for providing an increased blood concentration of a statin and/or active forms of the statin, relative to that resulting from the administration of an equivalent dose of the conventional immediate release formulations, comprising: a core and an outer coating that surrounds the core; the core comprising a statin and/or a pharmaceutically acceptable salt and/or ester thereof, and optionally at least one burst controlling agent and one disintegrant; and the coating comprising a water-insoluble hydrophobic carrier and a hydrophilic particulate matter.

In yet another embodiment, the present invention provides a delayed burst release formulation comprising a core and an outer coating that surrounds the core; the core comprising a statin, or a pharmaceutically acceptable salts and/or esters thereof excluding dihydroxy open-acid esters and salts of statins, and optionally at least one burst controlling agent and at least one disintegrant; and the outer coating comprising a water insoluble hydrophobic carrier and water-insoluble but hydrophilic particulate matter.

In yet another embodiment, the present invention provides a formulation for providing enhanced bioavailability of a statin and/or a pharmaceutically acceptable salt and/or ester thereof in a subject, comprising a delayed burst release formulation for oral administration comprising a core and an outer coating that surrounds the core; the core comprising a statin and/or a pharmaceutically acceptable salt and/or ester thereof, and optionally at least one burst controlling agent and at least one disintegrant; and the coating comprising a water-insoluble hydrophobic carrier and a hydrophilic particulate matter; characterized in that at least about 70% of the statin is released in vitro about one hour after the delayed burst release occurs.

In yet another embodiment, the present invention provides a formulation for release of statin and/or a pharmaceutically acceptable salt and/or ester thereof mainly in the colon of a subject, comprising: (a) a core that comprises an effective amount of statin and/or a pharmaceutically acceptable salt and/or ester thereof wherein the core contains at least one burst controlling agent and at least one disintegrant, and wherein the core is formed as a compressed tablet; and (b) an outer coating over the core, the outer coating comprising a water insoluble hydrophobic carrier and water-insoluble but hydrophilic particulate matter, contained in the carrier, that forms channels in the outer coating material upon contact with the colon medium, wherein the channels imbibe liquid and cause the at least one burst controlling agent to burst the coating, thereby providing delayed burst release of statin and/or a pharmaceutically acceptable salt and/or ester thereof after at least two hours followed by dispersion of statin and/or a pharmaceutically acceptable salt and/or ester thereof into the blood mainly through the colon over a period extending over at least twenty-four hours.

In yet another embodiment, the present invention provides a formulation that provides an increased amount of an active form of simvastatin and/or pharmaceutically acceptable salts and/or esters thereof circulating in the blood of a subject, the increased amount being determined relative to an amount provided by an equivalent dose of a conventional immediate release formulation, comprising: a delayed burst release formulation for oral administration comprising a core and an outer coating that surrounds the core; the core comprising simvastatin and/or a pharmaceutically acceptable salt and/or ester thereof, and optionally at least one burst controlling agent and at least one disintegrant; and the coating comprising a water-insoluble hydrophobic carrier and a hydrophilic particulate matter.

In yet another embodiment, the present invention provides a delayed, burst-release, pharmaceutical formulation for introducing a clinically effective amount of a hydroxy acid metabolite of a statin into the blood stream of a patient comprising a core and an outer coating that surrounds the core, the core comprising a statin, or pharmaceutically acceptable salts and/or esters thereof, and optionally at least one burst controlling agent and at least one disintegrant; and the outer coating comprising a water insoluble hydrophobic carrier and water-insoluble but hydrophilic particulate matter, wherein the formulation releases the statin in the lower gastrointestinal tract.

In yet another embodiment, the present invention provides a delayed, burst release, pharmaceutical formulation comprising a core and an outer coating that surrounds the core, the core comprising a statin, or pharmaceutically acceptable salts and/or esters thereof, and optionally at least one burst controlling agent and at least one disintegrant; and the outer coating comprising a water insoluble hydrophobic carrier and insoluble but hydrophilic particulate matter, wherein the formulation releases the statin in the lower gastrointestinal tract, and provides clinically effective amounts of a hydroxy acid metabolite of the statin into the blood stream of a patient.

In yet another embodiment, the present invention provides a delayed, burst-release, pharmaceutical formulation comprising a core and an outer coating that surrounds the core, the core comprising a statin, or pharmaceutically acceptable salts and/or esters thereof, and optionally at least one burst controlling agent at least one disintegrant; and the outer coating comprising a water-insoluble hydrophobic carrier and water-insoluble but hydrophilic particulate matter, wherein the formulation releases the statin in the lower gastrointestinal tract, and provides a clinically effective blood level of a hydroxy acid metabolite of the statin in the peripheral circulation of a patient.

In yet another embodiment, the present invention provides a pharmaceutical composition for oral administration, comprising a delayed, burst-release, pharmaceutical formulation comprising a core and an outer coating that surrounds the core, the core comprising a statin, or pharmaceutically acceptable salts and/or esters thereof, and optionally at least one burst controlling agent and at least one disintegrant; and the outer coating comprising a water-insoluble hydrophobic carrier and water-insoluble but hydrophilic particulate matter, wherein the formulation releases the statin in the lower gastrointestinal tract, wherein the statin is present in a decreased dosage amount of up to about 60% as compared to an immediate release formulation, yet provides at least similar pharmaceutical efficacy.

It is apparent to a person skilled in the art that the present invention is not limited to the particular delayed burst release formulations described herein, and that any delayed burst release formulation of statins, their pharmaceutically acceptable salts or esters or their active forms, that 1) provides an increased amount of the statin to the circulation of a subject; 2) provides enhanced bioavailability of the statin in a subject; 3) provides fast release of the statin in a subject; 4) includes a reduced amount of the statin (preferably up to 60%) as compared to an immediate release formulation of the statin, while providing a substantially similar therapeutic effect to the immediate release formulation; 5) releases substantially no statin in vitro for at least about 1 hour, preferably for at least about 1.5 hours, more preferably for at least about 2 hours; 6) releases at least about 70% of the statin in vitro about one hour after the delayed burst release occurs; 7) provides substantially zero in vivo blood plasma concentrations of the statin for at least about 1.5 hours or 2 hours after oral administration; and 8) provides therapeutically effective amounts of the statin at least about 12 hours, preferably for about 24 hours after the burst release occurs, is encompassed within the broad scope of the present invention.

Thus, further contemplated within the broad scope of the invention is a delayed burst release oral formulation for localized release of a statin in the gastrointestinal tract of a subject, characterized in that the in vivo blood plasma concentration of the statin or a pharmaceutically acceptable salt or ester thereof in the subject is substantially zero for at least about 1.5 hours after oral administration. In another embodiment, the in vivo blood plasma concentration of the statin or a pharmaceutically acceptable salt or ester thereof in the subject is substantially zero for at least about 2 hours after oral administration.

In another aspect, the present invention provides a delayed burst release oral formulation that provides an increased amount of a statin, a pharmaceutically acceptable salt or ester thereof, or an active form thereof to the circulation of a subject, compared to a substantially similar dose of an immediate release formulation of the statin.

In yet another aspect, the present invention provides a delayed burst release oral formulation that provides enhanced bioavailability of a statin, a pharmaceutically acceptable salt or ester thereof, or an active form thereof in a subject, compared to a substantially similar dose of an immediate release formulation of the stain.

In yet another aspect, the present invention provides a delayed burst release oral formulation for localized release of a statin in the gastrointestinal tract of a subject, wherein the statin is present in a decreased dosage amount of up to about 60% as compared to an immediate release formulation of the statin, while providing a substantially similar therapeutic effect to the immediate release formulation.

In yet another aspect, the present invention provides a delayed burst release oral formulation for localized release of a statin in the gastrointestinal tract of a subject characterized in that the formulation releases substantially no statin in vitro preferably for at least about 1 hour, or for at least about 1.5 hours, or for at least about 2 hours.

In yet another aspect, the present invention provides a delayed burst release oral formulation for localized release of a statin in the gastrointestinal tract of a subject, characterized in that at least about 70% of the statin is released in vitro about one hour after the delayed burst release occurs.

In yet another aspect, the present invention provides a delayed burst release oral formulation for localized release of a statin in the gastrointestinal tract of a subject the core, characterized in that the formulation provides therapeutically effective amounts of the statin, a pharmaceutically acceptable salt or ester thereof or an active form thereof in the subject for at least about 12 hours after the burst release occurs, preferably for at least about 24 hours after the burst release occurs.

The core of the formulations of the present invention contains a statin, which is preferably selected from simvastatin, lovastatin, mevastatin, pravastatin, fluvastatin, atorvastatin; cerivastatin and pitavastatin or pharmaceutically acceptable salts, esters, metabolites, hydrates, polymorphs, or crystals thereof. According to one currently preferred embodiment the statin is simvastatin. According to another currently preferred embodiment the statin is pitavastatin. According to other preferred embodiments the statin is lovastatin or atorvastatin.

The term “statin” as used herein includes also pharmaceutically acceptable salts, esters, metabolites, hydrates, polymorphs, or crystals thereof, and includes both statins in the lactone form or in the corresponding open dihydroxy acid.

The term “simvastatin” includes simvastatin and pharmaceutically acceptable salts, esters, metabolites, hydrates, polymorphs, or crystals thereof, in the lactone form or in the corresponding open dihydroxy acid, as disclosed for example in U.S. Pat. No. 4,444,784, which is hereby incorporated by reference in its entirety as if fully set forth herein.

The term “lovastatin” includes lovastatin and pharmaceutically acceptable salts, esters, metabolites, hydrates, polymorphs, or crystals thereof, in the lactone form or in the corresponding open dihydroxy acid, as disclosed for example in U.S. Pat. No. 4,231,938, which is hereby incorporated by reference in its entirety as if fully set forth herein.

The term “mevastatin” includes mevastatin and pharmaceutically acceptable salts, esters, metabolites, hydrates, polymorphs, or crystals thereof, in the lactone form or in the corresponding open dihydroxy acid, as disclosed for example in U.S. Pat. No. 3,671,523, which is hereby incorporated by reference in its entirety as if fully set forth herein.

The term “pravastatin” includes pravastatin and pharmaceutically acceptable salts, esters, metabolites, hydrates, polymorphs, or crystals thereof, in the lactone form or in the corresponding open dihydroxy acid, as disclosed for example in U.S. Pat. No. 4,346,227, which is hereby incorporated by reference in its entirety as if fully set forth herein.

The term “fluvastatin” includes fluvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,354,772, which is hereby incorporated by reference in its entirety as if fully set forth herein.

The term “atorvastatin” includes atorvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,273,995, which is hereby incorporated by reference in its entirety as if fully set forth herein.

The term “rivastatin” includes rivastatin and pharmaceutically acceptable salts, esters, metabolites, hydrates, polymorphs, or crystals thereof, in the lactone form or in the corresponding open dihydroxy acid, as disclosed for example in U.S. Pat. No. 5,177,080, which is hereby incorporated by reference in its entirety as if fully set forth herein.

The term “pitavastatin” (“nisvastatin”) includes pitavastatin and pharmaceutically acceptable salts, esters, metabolites, hydrates, polymorphs, or crystals thereof, in the lactone form or in the corresponding open dihydroxy acid, as disclosed for example in U.S. Pat. No. 5,011,930, U.S. Pat. No. 5,872,130, U.S. Pat. No. 5,856,336, which are hereby incorporated by reference in their entirety as if fully set forth herein.

As used herein, the term “active form” refers to any form of a molecule that can function as an HMG-CoA reductase inhibitor including the active ingredient administered and any active derivative resulting from metabolism or otherwise obtained from the parent molecule that can act as an HMG-CoA reductase. For example in the case of simavastatin marketed under the tradename ZOCOR® the known active forms include α-hydroxyacid of simvastatin and its 6β-hydroxy, 6β-hydroxymethyl, and 6β-exomethylene derivatives. The term “metabolite”, as used herein, includes any active form of the statin as described herein.

Suitable pharmaceutically acceptable salts include but are not limited to inorganic salts such as, for example, sodium, potassium, ammonium, calcium, and the like.

The doses of the statins to be used in the formulations of the present invention can be determined by a person of skill in the art, and will vary depending on the statin being used, the patient, and the condition being treated. Typical known therapeutic doses for each of the statins can be used as a guide to determine the appropriate dose to be used herein. As mentioned above, the formulations of the present invention preferably contain a reduced dose of the statin, as compared with the corresponding conventional formulation, preferably up to about 60% of the conventional dose for each statin.

The formulation is optionally in the form of a coated tablet. Alternatively, the formulation may be in the form of a pellet, microparticles, an agglomerate, a capsule, a pill or any other solid dosage form known to a person of skill in the art.

The combination of the selected materials for the core and outer layer, and the relative concentrations thereof, as well as the thickness of the core matrix and outer layer, determine both the lag time, which is defined herein as the time, post administration, when the release starts, as well as the rate of release of the drug.

Burst release is the preferred mechanism for release of the active ingredient in the formulations of the present invention. Without wishing to be limited by a single hypothesis or theory, the preferred embodiment of the formulation according to the present invention preferably includes a core which contains a swellable material, covered by a coating which includes a water insoluble, water permeable agent, through which water enters the core. The swellable material in the core then swells and bursts the coating, after which the core preferably disintegrates slowly or otherwise releases the active ingredient.

Release of the active agent of the present formulation preferably occurs within about 2-6 hours of oral administration, for example within 3 hours after oral administration or within 4 hours after oral administration, with a slightly longer delay occurring with the enteric coated formulations.

The Core

The core optionally includes a burst controlling agent. Preferably, the burst controlling agent comprises a water insoluble polymer for controlling the rate of penetration of water into the core and raising the internal pressure (osmotic pressure) inside the core. Such a burst controlling agent is preferably able to swell upon contact with liquid, e.g., bodily fluids.

Preferred but non-limiting examples of the water insoluble polymer include cross-linked polysaccharides, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

Examples of the cross-linked polysaccharide include but are not limited to insoluble metal salts or cross-linked derivatives of alginate, pectin, xantham gum, guar gum, tragacanth gum, and locust bean gum, carrageenan, metal salts thereof, and covalently cross-linked derivatives thereof.

Examples of the modified cellulose include but are not limited to cross-linked derivatives of hydroxypropylcellulose, hydroxypropylmethylcellulose, hydroxyethylcellulose, methylcellulose, carboxymethylcellulose, and metal salts of carboxymethylcellulose.

In accordance with certain currently preferred embodiment, the water insoluble polymer is calcium pectinate, microcrystalline cellulose or a combination thereof.

Optionally, the core also optionally contains one or more of an absorption enhancer, a binder, a disintegrant, and optionally at least one other excipient or a combination thereof.

Examples of a binder include but are not limited to Povidone (PVP: polyvinyl pyrrolidone), low molecular weight HPC (hydroxypropyl cellulose), low molecular weight HPMC (hydroxypropyl methylcellulose), low molecular weight carboxymethyl cellulose, ethylcellulose, gelatin, polyethylene oxide, acacia, dextrin, magnesium aluminum silicate, starch, and polymethacrylates. More preferably, the binder is Povidone.

Examples of a disintegrant include but are not limited to, Croscarmellose sodium (cross-linked sodium carboxymethyl cellulose), Crospovidone (cross-linked PVP), sodium carboxymethyl starch (sodium starch glycolate), pregelatinized starch (starch 1500), microcrystalline starch, water insoluble starch, calcium carboxymethyl cellulose, magnesium aluminum silicate (Veegum) or a combination thereof. Most preferably, the disintegrant is croscarmellose sodium.

According to preferred embodiments of the present invention, the core further comprises a synergist agent (sequestrate). Preferably, the sequestrate is selected from the group consisting of citric acid and ascorbic acid.

According to preferred embodiments of the present invention, the core further comprises a chelating agent. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetate.

According to some embodiments of the present invention, the core further comprises both a chelator and a synergistic agent (sequestrate). Without wishing to be limited by a single hypothesis or theory, chelating agents and sequestrates may optionally be differentiated as follows. A chelating agent, such as citric acid is intended to help in chelation of trace quantities of metals thereby assisting to prevent the loss of the active ingredient(s), such as simvastatin, by oxidation. A sequestrate such as ascorbic acid, optionally and preferably has several hydroxyl and/or carboxylic acid groups, which can provide a supply of hydrogen for regeneration of the inactivated antioxidant free radical. A sequestrate therefore preferably acts as a supplier of hydrogen for rejuvenation of the primary antioxidant.

According to preferred embodiments of the present invention, the core further comprises an antioxidant. Preferably, the antioxidant is selected from the group consisting of 4,4(2,3-dimethyl tetramethylene dipyrochatechol), Tocopherol-rich extract (natural vitamin E), □-tocopherol (synthetic Vitamin E), □-tocopherol, □-tocopherol, □-tocopherol, Butythydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate(Calcium disodium EDTA), Lactic acid, Trihydroxy butyrophenone and Thiodipropionic acid. According to a currently preferred embodiment, the antioxidant is BHA.

According to preferred embodiments of the present invention, the core further comprises a stabilizer. Preferably, the stabilizer can be a basic substance which can elevate the pH of an aqueous solution or dispersion of the formulation to at least about 6.8. Examples of such basic substances include but not limited to antiacids such as magnesium aluminometasilicate, magnesium aluminosilicate, magnesium aluminate, dried aluminum hydroxide, synthetic hydrotalcite, synthetic aluminum silicate, magnesium carbonate, precipitated calcium carbonate, magnesium oxide, aluminum hydroxide, and sodium hydrogencarbonate, and mixtures thereof; and pH-regulator agents such as L-arginine, sodium phosphate, disodium hydrogen phosphate, sodium dihydrogenphosphate, potassium phosphate, dipotassium hydrogenphosphate, potassium dihydrogenphosphate, disodium citrate, sodium succinate, ammonium chloride, and sodium benzoate and mixtures thereof. The basic substance can be selected from the group consisting of an inorganic water-soluble or inorganic water-insoluble compound.

Examples of inorganic water-soluble basic substance includes but are not limited to carbonate salt such as sodium or potassium carbonate, sodium bicarbonate, potassium hydrogen carbonate, phosphate salts selected from, e.g., anhydrous sodium, potassium or calcium dibasic phosphate, trisodium phosphate, alkali metal hydroxides, selected from sodium, potassium, or lithium hydroxide, and mixtures thereof. Sodium bicarbonate advantageously serves to neutralize acid groups in the composition in the presence of moisture that may adsorb onto particles of the composition during storage. The calcium carbonate exerts a buffering action in the stored composition, without apparent effect on drug release upon ingestion. It has further been discovered that the carbonate salts sufficiently stabilize the drug substance such that conventional water-based preparative techniques, e.g. trituration with water or wet granulation, can be utilized to prepare stabilized compositions of the invention.

Examples of inorganic water-insoluble basic substance include but not limited to suitable alkaline compounds capable of imparting the requisite basicity, include certain pharmaceutically acceptable inorganic compounds commonly employed in antiacid compositions e.g., magnesium oxide, magnesium hydroxide, or magnesium carbonate, magnesium hydrogen carbonate, aluminum or calcium hydroxide or carbonate, composite aluminum-magnesium compounds, such as magnesium aluminum hydroxide, silicate compound such as magnesium aluminum silicate (Veegum F), magnesium aluminometasilicate (Nesulin FH2), magnesium aluminosilicate (Nisulin A); as well as pharmaceutically acceptable salts of phosphoric acid such as tribasic calcium phosphate; and mixtures thereof.

Other optional ingredients for the core include, but are not limited to, one or more of a filler, a flow regulating agent and a lubricant. Examples of suitable fillers include but are not limited to, microcrystalline cellulose (e.g., Avicel™), starch, lactitol, lactose, dibasic calcium phosphate or any other type of suitable inorganic calcium salt and sucrose, or a combination thereof. A currently preferred filler is lactose monohydrate.

Examples of suitable lubricants include but are not limited to, stearate salts such as magnesium stearate, calcium stearate, and sodium stearate; stearic acid, talc, sodium stearyl fumarate, and compritol (glycerol behenate), corola oil, glyceryl palmitostearate, hydrogenated vegetable oil, magnesium oxide, mineral oil, poloxamer, polyethylene glycol, polyvinyl alcohol, sodium benzoate, talc, sodium stearyl fumarate, compritol (glycerol behenate) and sodium lauryl sulfate (SLS) or a combination thereof. A currently preferred lubricant is magnesium stearate.

Examples of suitable flow regulating agents include but are not limited to, colloidal silicon dioxide and aluminum silicate. A currently preferred flow regulating agent is colloidal silicon dioxide.

The core can also optionally include a buffering agent such as, for example, an inorganic salt compound and an organic alkaline salt compound. Preferably, the buffering agent is selected from the group consisting of potassium bicarbonate, potassium citrate, potassium hydroxide, sodium bicarbonate, sodium citrate, sodium hydroxide, calcium carbonate, dibasic sodium phosphate, monosodium glutamate, tribasic calcium phosphate, monoethanolamine, diethanolamine, triethanolamine, citric acid monohydrate, lactic acid, propionic acid, tartaric acid; fumaric acid, malic acid, and monobasic sodium phosphate.

According to specific embodiments, the core further includes a stabilizer. Preferably, the stabilizer comprises at least one of butyl hydroxyanisole, ascorbic acid and citric acid. According to specific embodiments, the hardness enhancing agent is microcrystalline cellulose.

Optionally, the core further comprises a preservative. Preferably, the preservative is selected from the group consisting of antioxidants, dipotassium edentate, disodium edentate, edetate calcium disodium, edetic acid, fumaric acid, malic acid, maltol, sodium edentate, and trisodium edetate.

The Coating

According to a second aspect of the invention, the coating surrounding the drug containing core comprises water-insoluble hydrophilic particulate matter embedded in a water-insoluble hydrophobic carrier. Preferably, the outer coating, is not pH sensitive.

The water-insoluble hydrophobic carrier is preferably a water insoluble polymer. Examples of suitable hydrophobic carriers include but are not limited to dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, said polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

The water-insoluble, hydrophilic particulate matter in the outer coating is preferably a water insoluble but permeable polymer. Non-limiting examples of such polymers include a water insoluble cross-linked polysaccharide, a water insoluble cross-linked protein, a water insoluble cross-linked peptide, water insoluble cross-linked gelatin, water insoluble cross-linked hydrolyzed gelatin, water insoluble cross-linked collagen, water insoluble cross linked polyacrylic acid, water insoluble cross-linked cellulose derivatives, water insoluble cross-linked polyvinyl pyrrolidone, micro crystalline cellulose, insoluble starch, micro crystalline starch and a combination thereof. According to one currently preferred embodiment, the water insoluble particulate matter is micro crystalline cellulose. According to another currently preferred embodiment, the water-insoluble hydrophilic particulate matter comprises a mixture of Avicel (microcrystalline cellulose) and ethocel.

The outer coating can optionally include at least one plasticizer. Examples of suitable plasticizers include but are not limited to, cetyl alcohol, dibutyl phthalate, diethyl phthalate, dibutyl sebacate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol, polyethylene glycol, propylene glycol and sorbitol. Combinations of these plasticizers are also contemplated. Typically, the amount of plasticizer is in a range of from about 0 to about 50% weight per weight of the water insoluble polymer in the film coat.

In addition or alternatively, a stiffening agent such as cetyl alcohol can also be used. The outer coating or the core or both can also optionally contain at least one of a wetting agent, suspending agent, surfactant, and dispersing agent, or a combination thereof.

Examples of suitable wetting agents include, but are not limited to, poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, docusate sodium.

Examples of suitable suspending agents include but are not limited to, alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), polyvinyl pyrrolidone (PVP), propylene glycol alginate, sodium alginate, sorbitan fatty acid esters, and tragacanth.

Examples of suitable surfactants include but are not limited to, anionic surfactants such as docusate sodium and sodium lauryl sulfate; cationic, such as cetrimide; nonionic, such as polyoxyethylene sorbitan fatty acid esters (polysorbates) and sorbitan fatty acid esters.

Examples of suitable dispersing agents include but are not limited to, poloxamer, polyoxyethylene sorbitan fatty acid esters (polysorbates) and sorbitan fatty acid esters.

The content of the wetting agent, surfactant, dispersing agent and suspending agent can range in an amount of from about 0 to about 30% of the weight of the film coat of the formulation. A particularly preferred embodiment of the present invention includes crospovidone (cross-linked PVP) or croscarmellose, calcium pectinate, microcrystalline cellulose, ethylcellulose, polyvinyl pyrrolidone (PVP), colloidal silicon dioxide, butyl hydroxyanisole, citric acid, ascorbic acid, and magnesium stearate in the core. The coating for this embodiment preferably includes ethyl cellulose, cetyl alcohol, microcrystalline cellulose or calcium pectinate (CaP).

Optionally, an enteric coating can be applied to these coated cores. The enteric coating can comprise any suitable enteric coating material, such as hydroxypropylmethyl cellulose phthalate, polyvinyl acetate phthalate, cellulose acetate phthalate, hydroxypropylmethyl cellulose acetate succinate, poly(methacrylic acid, methyl methacrylate) 1:1 (Eudragit L-100), poly(methacrylic acid, ethyl acrylate) 1:1 (Eudragit L-30D 55), alginic acid, and sodium alginate.

The outer enteric coating may further comprise a plasticizer. The plasticizer preferably includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

According to a preferred embodiment, the enteric coating comprises methacrylic acid copolymer, triethyl citrate and talc.

The formulations of the present invention can be prepared in accordance with any formulation processes known to a person of skill in the art. Such methods include a wet granulation process, a dry mix process, a direct compression process, etc. In one embodiment, several of the core ingredients are mixed by a wet granulation process to form a granulate, which is then dried and dry-mixed with several other ingredients to form the core, which is then coated with a coating composition according to the present invention.

Therapeutic Uses:

The formulations of the present invention are capable of providing a therapeutically effective amount of a statin a pharmaceutically acceptable salt or ester thereof or an active form thereof to a subject, for an extended period of time after the burst release occurs. The formulations according to the present invention have increased efficacy and to provide at least a similar, if not greater, pharmaceutical effect with the active ingredient, using a significantly decreased dosage amount as compared with conventional formulations known in the art regarding reduce of elevated total cholesterol, low density lipoprotein cholesterol, apolipoprotein B, triglycerides and increase of high density lipoprotein cholesterol. Preferably, the formulations of the present invention contain the statin in an amount that is up to about 60% as compared to an immediate release formulation, yet provides at least similar pharmaceutical efficacy. Thus, the novel formulations of the present invention are more effective than conventional statin formulations currently in use, and can be used for treating high cholesterol, ischemic heart disease and myocardial infarction, or any other disease or condition for which statins are indicated. The formulations of the present invention may even lead to new indications for the use of delayed burst release of simvastatin and can be used in new populations of patients in which the conventional statin formulations are not used for at present. The formulations of the present invention comprise at least one statin in a decreased dosage amount of up to about 50% as compared to an immediate release formulation of the statin, while providing a substantially equivalent effect of lowering of LDL as a full dosage of the immediate release formulation.

Thus in one aspect, the present invention relates to a method for providing a therapeutically effective amount of a statin, a pharmaceutically acceptable salt or ester thereof or an active form thereof to a subject, comprising orally administering to the subject a delayed burst release formulation, according to the present invention.

In another aspect, the present invention relates to a method for providing enhanced bioavailability of a statin, a pharmaceutically acceptable salt or ester thereof or an active form thereof to a subject, comprising orally administering to the subject a delayed burst release formulation according to the present invention.

In yet another aspect, the present invention provides a method of providing fast release of a statin a pharmaceutically acceptable salt or ester thereof or an active form thereof in the gastrointestinal tract of a subject, comprising orally administering to the subject a delayed burst release formulation according to the present invention.

In another aspect, the present invention provides a method for introducing clinically effective amounts of a hydroxy acid metabolite of a statin into the blood stream of a patient comprising administering a delayed, burst-release, pharmaceutical formulation according to the present invention, wherein the formulation releases the statin in the lower gastrointestinal tract, and provides clinically effective amounts of a hydroxy acid metabolite of the statin into the blood stream of a patient.

In another aspect, the present invention provides a method for administering a reduced amount of a statin to a subject, comprising: administering a formulation to the subject containing the statin in an amount that is up to about 60% as compared to an immediate release formulation, yet provides at least similar pharmaceutical efficacy.

Of course, as mentioned above, the methods of the present invention are not limited to the delayed burst release formulations described herein. Any delayed burst release formulation comprising a statin which provides an increased amount of the statin to the circulation of a subject; provides enhanced bioavailability of the statin in a subject; provides fast release of the statin in a subject; includes a reduced amount of the statin (preferably up to 60%) as compared to an immediate release formulation of the statin, while providing a substantially similar therapeutic effect to the immediate release formulation; releases substantially no statin in vitro for at least about 1 hour; releases at least about 70% of the statin in vitro about one hour after the delayed burst release occurs; provides substantially zero blood levels of the statin for a period of at least about 1.5 hours or 2 hours after oral administration, and provides therapeutically effective amounts of the statin at least about 12 hours, preferably for about 24 hours after the burst release occurs, can be used in the methods of the present invention.

Thus, further included within the broad scope of the present invention, is a method for providing a therapeutically effective amount of a statin, a pharmaceutically acceptable salt or ester thereof or an active form thereof to a subject, comprising orally administering to the subject a delayed burst release formulation comprising a statin, a pharmaceutically acceptable salt or ester thereof or an active form thereof. Preferably, the therapeutic amount is maintained over a period of at least 12 hours, preferably at least about 24 hours after the burst release occurs

In yet a further aspect, the present invention relates to a method for providing enhanced bioavailability of a statin, a pharmaceutically acceptable salt or ester thereof or an active form thereof to a subject, comprising orally administering to the subject a delayed burst release formulation comprising a statin, a pharmaceutically acceptable salt or ester thereof or an active form thereof.

In yet a further aspect, the present invention provides a method of providing fast release of a statin a pharmaceutically acceptable salt or ester thereof or an active form thereof in the gastrointestinal tract of a subject, comprising orally administering to the subject a delayed burst release formulation comprising a statin, a pharmaceutically acceptable salt or ester thereof or an active form thereof.

In yet a further aspect, the present invention provides a method for providing a therapeutically effective amount of a hydroxy acid metabolite of a statin, a pharmaceutically acceptable salt or ester thereof to a subject, by administering to the subject a delayed burst release formulation comprising a statin, a pharmaceutically acceptable salt or ester thereof or an active form thereof. Preferably, the therapeutic amount is maintained over a period of at least 12 hours, preferably at least about 24 hours after the burst release occurs

In a further aspect, the present invention provides a method of providing an increased amount of a statin, a pharmaceutically acceptable salt or ester thereof or an active form thereof to a subject, compared to a substantially similar dose of an immediate release formulation of the statin, comprising orally administering to the subject a delayed burst release formulation comprising the statin, a pharmaceutically acceptable salt or ester thereof.

In yet another aspect, the present invention provides a delayed burst release method for providing an increased amount of a statin and/or active forms of the statin circulating in the blood of a subject, relative to that resulting from the administration of an equivalent dose of a conventional immediate release formulation to the subject, comprising administering a formulation to the subject that comprises: a core and an outer coating that surrounds the core, the core comprising a statin and/or a pharmaceutically acceptable salt and/or ester thereof, at least one burst controlling agent and at least one disintegrant; and the coating comprising a water-insoluble hydrophobic carrier and a hydrophilic particulate matter.

The following examples are presented in order to more fully illustrate certain embodiments of the invention. They should in no way, however, be construed as limiting the broad scope of the invention. One skilled in the art can readily devise many variations and modifications of the principles disclosed herein without departing from the scope of the invention.

### EXAMPLES

The following examples are illustrative implementations of the methods and compositions according to the present invention using simvastatin as an exemplary statin compound. Part I relates to the formulations and in vitro data obtained with these formulations; Part II relates to in vivo data obtained with formulations according to the present invention; and Part III relates to an efficacy study using the formulations of the present invention.

Part 1—Formulations and In Vitro Data

### Example 1

### A 16 mg Simvastatin DBR Tablet

A. Core

| Excipient | mg/tablet | % |
|---|---|---|
| Wet granulation mixture | | |
| simvastatin | 16.00 | 5.33 |
| lactose monohydrate 100M | 20.00 | 6.67 |
| croscarmellose sodium | 1.30 | 0.43 |
| microcrystalline cellulose PH 101 | 19.55 | 6.52 |
| ascorbic acid | 3.00 | 1.00 |
| Povidone K-30 (polyvinylpyrrolidone) | 3.10 | 1.03 |
| citric acid anhydrous | 1.50 | 0.50 |
| butyl hydroxyanisole | 0.05 | 0.02 |
| Dry blend mixture | | |
| microcrystalline cellulose PH 102 | 221.60 | 73.87 |
| croscarmellose sodium | 6.00 | 2.00 |
| colloidal silicon dioxide | 6.00 | 2.00 |
| magnesium stearate | 1.90 | 0.63 |
| Total | 300.00 | 100.00 |

B. Time Controlled Release System (TCDS) Coating

| Excipient | mg/tablet |
|---|---|
| Microcrystalline cellulose (Avicel PH 102) | 21.9 |
| Ethylcellulose (Ethocel 20) | 14.6 |
| cetyl alcohol | 1.5 |
| Total | 38.0 |

### Example 2

### A 16 Mg Simvastatin DBR Tablet

Core as for Example 1

| | |
|---|---|
| Excipient | mg/tablet |

A. TCDS coating

| | |
|---|---|
| microcrystalline cellulose (Avicel PH 102) | 19.6 |
| Ethyl cellulose Ethocel 20 | 13.1 |
| cetyl alcohol | 1.3 |
| Total | 34.0 |

B. Enteric coating

| | |
|---|---|
| poly(methacrylic acid, ethyl acrylate)1:1 | 20.0 |
| compolymer (Eudragit L30D-55) | |
| triethyl citrate | 4.0 |
| talc | 2.0 |
| Total | 26.0 |

### Example 3

### A 40 Mg Simvastatin DBR Tablet

A. Core
The core of Simvastatin 40 mg DBR Tablets
Core

| Excipient | mg/tab | % |
|---|---|---|
| Wet granulation mixture | | |
| Simvastatin | 40.00 | 12.50 |
| Lactose monohydrate 100M | 50.00 | 15.63 |
| Croscarmellose Sodium | 3.20 | 1.00 |
| Microcrystalline cellulose (Avicel PH 101) | 48.90 | 15.28 |
| Ascorbic Acid | 7.50 | 2.34 |
| Polyvinyl pyrrolidone (Povidone K-30) | 7.70 | 2.41 |
| Citric Acid Anhydrous | 3.75 | 1.17 |
| Butyl Hydroxyanisole | 0.12 | 0.04 |
| Dry blend mixture | | |
| Microcrystalline cellulose (Avicel PH 102) | 144.00 | 45.00 |
| Croscarmellose Sodium | 6.40 | 2.00 |
| Colloidal Silicon Dioxide | 6.40 | 2.00 |
| Magnesium stearate | 2.00 | 0.63 |
| Total | 320.00 | 100.00 |

B. TCDS coating

| Excipient | mg/tablet |
|---|---|
| Microcrystalline cellulose (Avicel PH 102) | 23.1 |
| Ethyl cellulose (Ethocel 20) | 15.4 |
| cetyl alcohol | 1.5 |
| total | 40.0 |

### Example 4

### A 10 Mg Simvastatin DBR Tablet—

| Excipient | mg/tab | % |
|---|---|---|
| Core | | % of core |
| Simvastatin | 10.00 | 3.33% |
| Microcrystalline cellulose | 21.00 | 7.00% |
| Lactose monohydrate | 27.00 | 9.00% |
| Butyl Hydroxyanysole (BHA) | 0.12 | 0.04% |
| Citric acid | 3.75 | 1.25% |
| Ascorbic acid | 7.50 | 2.50% |
| Polyvinyl pyrrolidone (Povidone) | 2.20 | 0.73% |
| Croscarmellose sodium | 1.46 | 0.49% |
| Total Granulate | 73.03 | 24.34% |
| | Water + | |
| Granulation solution | IPA | |
| Croscarmellose sodium | 6.00 | 2.00% |
| Microcrystalline cellulose | 213.20 | 71.06% |
| Microcrystalline cellulose | | |
| Silica colloidal anhyd. | 6.00 | 2.00% |
| Magnesium stearate | 1.80 | 0.60% |
| Total core | 300.0 | 100.00% |
| TCDS Coating | (mg/tab) | % of Coat |
| Microcrystalline cellulose | 19.6 | 57.69% |
| Ethyl Cellulose | 13.1 | 38.46% |
| Cetyl alcohol | 1.3 | 3.85% |
| Total coated tablet | 334.0 | |
| Avicel/EC Rate/Coating weight (mg) | 34 | 60/40 |

Other formulations containing 10 mg simvastatin in a DBR formulation were prepared in a similar manner. In addition, different doses of simvastatin and other statins can be prepared in a similar manner, as discussed herein.

### Example 5

### An 8 Mg and a 4 Mg Pitavastatin DBR Tablet

A. Core

The core of Pitavastatin 8 mg and 4 mg DBR Tablets was composed of: Pitavastatin Calcium salts (8 mg and 4 mg), Lactose monohydrate, cross-linked polyvinylpyrrolidinone, microcrystalline cellulose PH 101, Povidone K-30, Magnesium alumino metasillicate (Nesulin FH2) and Magnesium Stearate.

The cores of example 5 were prepared by a granulation process. The granulate was dried over a fluidized bed granulator. Next, the second part of the core was dry-blended. Magnesium alumino metasilicate was mixed with an additional amount of cross-linked polyvinylpyrrolidinone and sieved by a mechanical sieve equipped with a 850 micron screen into the previously obtained granulation blend. The obtained mixture was blended and microcrystalline cellulose was added into the mixture.

Magnesium stearate, a lubricant, was passed through a mechanical sieve equipped with a 600 micron screen into the mixture and blended for 5 min. This last process resulted in the tabletting mixture. The tabletting mixture was then compressed with a Kilian tabletting press equipped with suitable punches set, such that the average weight of tablet would include a proper amount of the active material, with a hardness sufficient for subsequent coating.

B. TCDS Coating

The coating formulation was performed as described in the previous examples.

The formed cores were then coated with a Time Controlled Release System (TCDS®) coating that was prepared as follows. Ethyl cellulose was dissolved in ethanol to obtain a clear solution, to which a weighed quantity of plasticizer (cetyl alcohol) was added and mixed with the mechanical stirrer to complete dissolution. Sieved microcrystalline cellulose was added and stirred to obtain a homogeneous suspension, which was stirred during the whole coating process.

The coating was performed in a perforated pan coater, with an applied spraying pressure of 1.5-2.5 Bar. The tablets were coated until the weight of the coating was about 40-50 mg, and then dried.

### Example 6

### An 8 Mg and a 4 Mg Pitavastatin DBR Tablet

A. Core

The core of Pitavastatin 8 mg and 4 mg DBR Tablets was composed of: Pitavastatin Calcium salts (8 mg and 4 mg), Lactose monohydrate, pregelatinized starch (starch 1500), microcrystalline cellulose PH 101, hydroxypropylcellulose (low substitute), Magnesium alumino metasillicate (Nesulin FH2) and Magnesium Stearate.

The cores of example 6 were prepared by a granulation process. The granulate was dried over a fluidized bed granulator. Next, the second part of the core was dry-blended. Magnesium alumino metasilicate was mixed with an additional amount of pregelatinized starch (starch 1500) and sieved by a mechanical sieve equipped with a 850 micron screen into the previously obtained granulation blend. The obtained mixture was blended and microcrystalline cellulose was added into the mixture.

Magnesium stearate, a lubricant, was passed through a mechanical sieve equipped with a 600 micron screen into the mixture and blended for 5 min. This last process resulted in the tabletting mixture.

The tabletting mixture was then compressed with a Kilian tabletting press equipped with suitable punches set, such that the average weight of tablet would include a proper amount of the active material, with a hardness sufficient for subsequent coating.

B. TCDS Coating

The coating formulation was performed as described in the previous examples.

The formed cores were then coated with a Time Controlled Release System (TCDS®) coating that was prepared as follows. Ethyl cellulose was dissolved in ethanol to obtain a clear solution, to which a weighed quantity of plasticizer (cetyl alcohol) was added and mixed with the mechanical stirrer to complete dissolution. Sieved microcrystalline cellulose was added and stirred to obtain a homogeneous suspension, which was stirred during the whole coating process.

The coating was performed in a perforated pan coater, with an applied spraying pressure of 1.5-2.5 Bar. The tablets were coated until the weight of the coating was about 40-50 mg, and then dried.

The cores for Examples 1 and 3 were prepared by a granulation process. First, Povidone K-30, citric acid and butyl hydroxyanisole were dissolved in ethanol by using a mechanical stirrer to obtain a clear solution.

Simvastatin was mixed with lactose monohydrate 100M, microcrystalline cellulose PH 101, ascorbic acid and croscarmellose sodium (as disintegrator), the mixture was granulated by adding the granulation solution into the granulator. The granulate was dried over fluidized bed granulator. The dried granulation blend was milled to obtain the desired particle size distribution of the final granulation blend.

Next, the second part of the core was dry-blended. Colloidal silicon dioxide was mixed with an additional amount of croscarmellose sodium and sieved by a mechanical sieve equipped with a 850 micron screen into the previously obtained granulation blend. The obtained mixture was blended and microcrystalline cellulose was added into the mixture.

Magnesium stearate, a lubricant, was passed through a mechanical sieve equipped with a 600 micron screen into the mixture and blended for 5 min. This last process resulted in the tabletting mixture.

The tabletting mixture was then compressed with a Kilian tabletting press equipped with suitable punches set, such that the average weight of tablet would include a proper amount of the active material, with a hardness sufficient for subsequent coating.

The formed cores were then coated with a Time Controlled Release System (TCDS®) coating that was prepared as follows. Ethyl cellulose was dissolved in ethanol to obtain a clear solution, to which a weighed quantity of plasticizer (cetyl alcohol) was added and mixed with the mechanical stirrer to complete dissolution. Sieved microcrystalline cellulose was added and stirred to obtain a homogeneous suspension, which was stirred during the whole coating process.

The coating was performed in a perforated pan coater, with an applied spraying pressure of 1.5-2.5 Bar. The tablets were coated until the weight of the coating was about 40-50 mg, and then dried.

The core and TCDS coating for example 2 were prepared as described above for examples 1 and 3. The tablets were then subjected to enteric coating as follows. A weighed quantity of plasticizer (Triethyl citrate) was dissolved in purified water to obtain a clear solution, to which methacrylic acid copolymer was added and mixed. Talc was added and stirred to obtain a homogeneous suspension.

The coating was performed in perforated pan coater at temperature of the incoming air kept to 50° C. and applied spraying pressure of 1.5-2.5 Bar. The tablets were then dried

In Vitro Release Profile—Simvastatin Formulations Containing TCDS

The in vitro release of simvastatin from the above-referenced formulations was determined as follows. Each of six simvastatin tablets were inserted into an individual dissolution cell, each of which contained (for examples 1 and 2) 900 ml HCl (0.1 M). After one hour, the dissolution medium was changed to 900 ml buffer USP pH 7 with 0.5% Sodium Lauryl Sulphate (SLS). For example 3, the medium was 900 ml buffer USP pH 7 with 0.5% SLS throughout the dissolution test. The sample was stirred with a VanKel basket stirrer (Van Kel Inc., USA). Samples were automatically drawn from each dissolution cell to test tubes at various time points; for examples 1 and 2, samples were taken at 1.08 h, 1.25 h and then every 15 min, up to 2.5 h and then at 3 h, 4 h and 6 h. For example 3, samples were taken at 1 h, and every 30 min up to 3 h, then at 4.5 and 7 h). Samples were analyzed by a UV (ultraviolet) light detection (238 nm) and analysis device (HPLC). The amount of drug released was calculated according to a standard set of calculations that are known in the art.

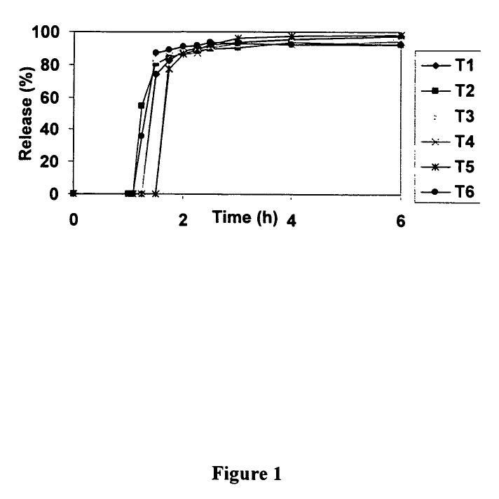

FIG. 1 and Table 1 show the in vitro dissolution profile for six different tablet batches (labeled T1-T6) of a formulation containing 16 mg simvastatin according to Example 1, which does not comprise an enteric coating. As can be seen, the burst release occurs after a lag of approximately 1.75 hours following administration, and the plateau is maintained for at least 4 hours.

TABLE 1

| | T1-T6 |
|---|---|
| | (mean |
| hours | release) |
| 0 | 0.0 |
| 1 | 0.0 |
| 1.08 | 0.0 |
| 1.25 | 15.0 |
| 1.5 | 54.2 |
| 1.75 | 84.6 |
| 2 | 88.1 |
| 2.25 | 89.4 |
| 2.5 | 91.4 |
| 3 | 93.0 |
| 4 | 94.4 |
| 6 | 95.0 |

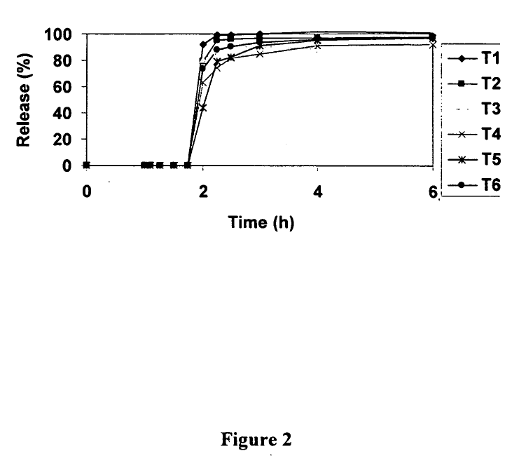

As seen in FIG. 2 and Table 2, relating to in vitro dissolution of a formulation containing 16 mg simvastatin according to Example 2, which includes an enteric outer coating, the lag time is slightly greater than that obtained with the formulation of Example 1, with the burst occurring after approximately 2 hours.

TABLE 2

| | T1-T6 |
|---|---|
| | (mean |
| hours | release) |
| 0 | 0.0 |
| 1 | 0.0 |
| 1.08 | 0.0 |
| 1.25 | 0.0 |
| 1.5 | 0.0 |
| 1.75 | 0.0 |
| 2 | 72.2 |
| 2.25 | 87.2 |
| 2.5 | 89.9 |
| 3 | 92.9 |
| 4 | 94.8 |
| 6 | 96.3 |

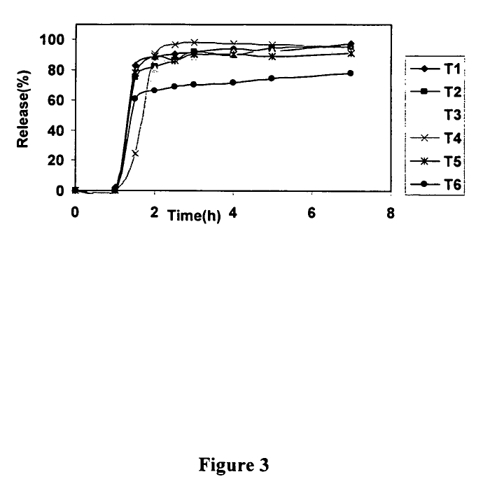

FIG. 3 and Table 3 show the in vitro dissolution profile for six different tablet batches (labeled T1-T6) of a formulation containing 40 mg simvastatin according to Example 3. As can be seen, the burst release occurs after a lag of approximately 2 hours following administration, and the plateau is maintained for at least 4 hours.

TABLE 3

| | T1-T6 |
|---|---|
| | (mean |
| hours | release) |
| 0 | 0 |
| 1 | 0.3 |
| 1.5 | 57.7 |
| 2 | 82.9 |
| 2.5 | 85.7 |
| 3 | 88.6 |
| 4 | 89.1 |
| 5 | 90.2 |
| 7 | 91.6 |

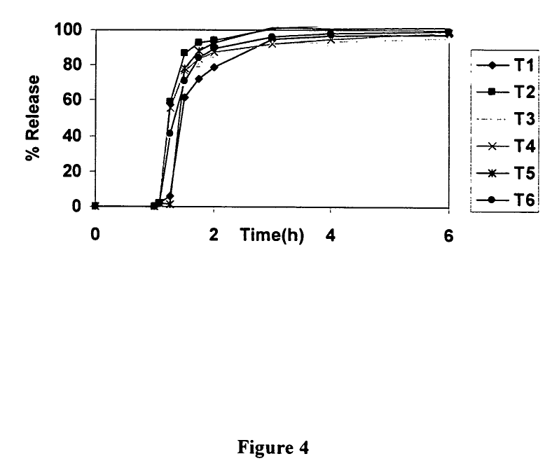

FIG. 4 and Table 4 show the in vitro dissolution profile for six different tablet batches (labeled T1-T6) of a formulation containing 10 mg simvastatin according to Example 4. As can be seen, the burst release occurs after a lag of approximately 1.5-1.75 hours following administration, and the plateau is maintained for at least 4 hours.

TABLE 4

| | T1-T6 |
|---|---|
| | (mean |
| hours | release) |
| 0 | 0.00 |
| 1 | 0.00 |
| 1.08 | 1.8 |
| 1.25 | 34.0 |
| 1.5 | 74.1 |
| 1.75 | 83.6 |
| 2 | 88.0 |
| 3 | 96.3 |
| 4 | 97.7 |
| 6 | 99.3 |

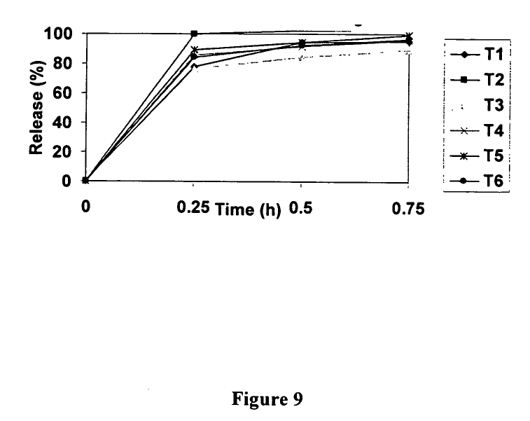

In comparison, as shown in FIG. 9 and Table 5, a simvastatin formulation containing no TCDS coating released over 80% of the active ingredient after 15 minutes, and virtually all of the simvastatin after about 45 minutes. In contrast, the formulations for the present invention provide delayed burst release of statins, with a lag period of at least about 1 hour, preferably at least about 1.5 hours, and more preferably at least about 2 hours.

TABLE 5

| | T1-T6 |
|---|---|
| | (mean |
| hours | release) |
| 0 | 0.0 |
| 0.25 | 85.5 |
| 0.50 | 93.3 |
| 0.75 | 96.4 |

In Vitro Release Profile Pitavastatin Calcium Salt-Containing TCDS

The in vitro release of pitavastatin from the above-referenced formulations was determined as follows. Each of six pitavastatin tablets were inserted into an individual dissolution cell, each of which contained 900 ml HCl (0.1 M). After one hour, the dissolution medium was changed to 900 ml buffer USP pH 6.8. The sample was stirred with a VanKel basket stirrer (Van Kel Inc., USA). Samples were automatically drawn from each dissolution cell to test tubes at various time points up to 7 hours. Samples were analyzed by High Performance Liquid Chromatography (HPLC). The amount of drug released was calculated according to a standard set of calculations that are known in the art. The results show an in vitro, release profile comparable to that of the simvastatin TCDS DBR tablets.

Part II—Bioavailability Study—Simvastatin-Containing TCDS DBR Tablet

Two randomized, pharmacokinetic pilot studies were undertaken to evaluate the bioavailability of test formulations of simvastatin, and also to determine the levels of the main metabolite, simvastatin hydroxy acid. For the first study, 40 mg simvastatin tablets were prepared according to Example 3, and for the second study two batches of 16 mg tablets were prepared according to Examples 1 (batch 1—no enteric coating) and 2 (batch 2—enteric coating).

In the first study, one 40 mg tablet was administered to fasting volunteers and blood samples were withdrawn pre-dose and at: 0.5, 1, 1.5, 2, 2.5, 3, 3.5, 4, 4.5, 5, 6, 8, 10, 12, 16, 24, 36, and 48 hours following the dose. A reference product purchased from Merck (Zocor 40 mg tablet Manufacturer Merck Frosst Canada & Co., Kirkland QC, Canada, Expiry date: December 2004) was used as control.

In the second study, two 16 mg test tablet formulations were administered to fasting volunteers according to the same conditions as in the first study.

Plasma concentrations of simvastatin or simvastatin hydroxy acid were determined using an LC/MS/MS analytical method. A concentration-time curve was constructed for each volunteer. The observed maximal concentration was recorded as Cmax. The area under the curve (AUC) and the time to maximal concentration (Tmax) were computed for each volunteer. The results were calculated for ten volunteers (n=10). All values below level of quantization (BLQ, 0.100 ng/nL) were set to zero for pharmacokinetic and statistical calculations.

Pharmacokinetic Parameters

TABLE 6
Mean plasma concentration simvastatin (ng/ml)

| Time | Simvastatin | Simvastatin | Simvastatin | Zocor |
|---|---|---|---|---|
| (hours) | 16 mg - batch 1 | 16 mg - batch 2 | 40 mg | 40 mg (Ref.) |
| 0.00 | 0.000 | 0.000 | 0.000 | 0.000 |
| 0.50 | 0.000 | 0.000 | 0.000 | 2.063 |
| 1.00 | 0.000 | 0.000 | 0.000 | 8.216 |
| 1.50 | 0.000 | 0.000 | 0.957 | 7.465 |
| 2.00 | 0.130 | 0.000 | 0.995 | 7.203 |
| 2.50 | 1.999 | 0.327 | 1.145 | 6.367 |
| 3.00 | 3.188 | 1.154 | 1.065 | 5.163 |
| 3.50 | 2.918 | 1.264 | 1.539 | 3.847 |
| 4.00 | 2.482 | 1.383 | 1.850 | 3.428 |
| 4.50 | 2.711 | 2.007 | 2.436 | 3.211 |
| 5.00 | 2.369 | 1.947 | 3.086 | 2.821 |
| 6.00 | 1.441 | 1.179 | 3.719 | 2.107 |
| 8.00 | 0.723 | 0.610 | 3.325 | 1.286 |
| 10.0 | 0.658 | 0.735 | 3.697 | 0.915 |
| 12.0 | 0.430 | 0.689 | 4.487 | 0.703 |
| 16.0 | 0.202 | 0.488 | 3.156 | 0.452 |
| 24.0 | 0.180 | 0.435 | 3.029 | 0.285 |
| 36.0 | 0.024 | 0.092 | 0.625 | 0.049 |
| 48.0 | 0.000 | 0.025 | 0.272 | 0.013 |

TABLE 7
Simvastatin hydroxy acid plasma concentration (ng/ml)

| | Simvastatin | Simvastatin | | |
|---|---|---|---|---|
| Time | 16 mg - | 16 mg - | Simvastatin | Zocor ® |
| (hours) | batch 1 | batch 2 | 40 mg | 40 mg (Ref.) |
| 0.00 | 0.000 | 0.000 | 0.000 | 0.000 |
| 0.50 | 0.000 | 0.000 | 0.024 | 0.110 |
| 1.00 | 0.000 | 0.000 | 0.018 | 0.509 |
| 1.50 | 0.000 | 0.000 | 0.178 | 0.720 |
| 2.00 | 0.088 | 0.000 | 0.156 | 1.116 |
| 2.50 | 1.193 | 0.023 | 0.287 | 1.255 |
| 3.00 | 1.782 | 0.267 | 0.511 | 1.569 |
| 3.50 | 2.037 | 0.556 | 0.614 | 1.626 |
| 4.00 | 2.162 | 0.825 | 0.740 | 1.717 |
| 4.50 | 2.777 | 1.381 | 1.162 | 2.261 |
| 5.00 | 2.188 | 1.416 | 0.864 | 1.562 |
| 6.00 | 1.788 | 1.272 | 0.802 | 1.152 |
| 8.00 | 1.444 | 0.917 | 0.766 | 0.890 |
| 10.0 | 1.392 | 1.132 | 1.063 | 0.876 |
| 12.0 | 1.037 | 1.025 | 1.100 | 0.604 |
| 16.0 | 0.734 | 1.081 | 0.932 | 0.262 |
| 24.0 | 0.412 | 0.775 | 1.103 | 0.082 |
| 36.0 | 0.164 | 0.219 | 0.514 | 0.037 |
| 48.0 | 0.044 | 0.028 | 0.167 | 0.013 |

Pharmakokinetic Parameters

TABLE 8
Simvastatin - First study

| | AUC_{(0-∞)} | AUC₍₀₋ₜ₎ | Cmax | Tmax | T½ |
|---|---|---|---|---|---|
| | (ng × hour/ml) | (ng × hour/ml) | (ng/ml) | (hours) | (hours) |
| Simvastatin 40 mg | 109.22 ± 61.10 | 97.6 ± 61.16 | 7.47 ± 3.55 | 8.10 ± 6.61 | 6.84 ± 2.60 |
| (Dexcel) | (31.15; 246.66) | (30.39; 246.12) | (1.47; 15.00) | (1.50; 24.00) | (3.01; 10.55) |
| | N = 8 | | | | N = 8 |
| Zocor 40 mg | 42.23 ± 26.43 | 40.38 ± 23.88 | 10.39 ± 6.23 | 1.75 ± 0.72 | 6.02 ± 1.90 |
| (Merck) | (6.48; 78.06) | (5.77; 76.62) | (4.85; 23.20) | (1.00; 3.00) | (3.07; 8.22) |
| | N = 9 | | | | N = 9 |
| Ratio* | 2.519 | 2.584 | 0.729 | | |
| (90% ANOVA C.I.) | (1.217; 5.217) | (1.488; 4.49) | (0.529; 1.004) | | |

TABLE 9
Simvastatin Hydroxyacid - First study

| | AUC_{(0-∞)} | AUC₍₀₋ₜ₎ | Cmax | Tmax | T½ |
|---|---|---|---|---|---|
| | (ng × hour/ml) | (ng × hour/ml) | (ng/ml) | (hours) | (hours) |
| Simvastatin 40 mg | 26.16 ± 5.46 | 33.64 ± 16.29 | 2.19 ± 1.26 | 13.10 ± 11.25 | 10.36 ± 6.10 |
| (Dexcel) | (20.46; 32.83) | (9.51; 60.12) | (0.73; 5.21) | (1.50; 36.00) | (3.05; 17.62) |
| | N = 4 | | | | N = 4 |
| Zocor 40 mg | 19.08 ± 8.77 | 15.93 ± 8.46 | 2.30 ± 1.14 | 5.40 ± 3.81 | 5.47 ± 3.61 |
| (Merck) | (8.00; 32.70) | (6.89; 31.77) | (1.13; 4.97) | (2.00; 16.00) | (2.37; 12.36) |
| | N = 8 | | | | N = 8 |
| Ratio* | | 2.121 | 0.915 | | |
| (90% ANOVA C.I.) | | (1.493; 3.014) | (0.684; 1.225) | | |

TABLE 10
simvastatin - Second study

| | AUC_{(0-∞)} | AUC₍₀₋ₜ₎ | Cmax | Tmax | T½ | LagTime |
|---|---|---|---|---|---|---|
| | (ng × hour/ml) | (ng × hour/ml) | (ng/ml) | (hours) | (hr.) | (hours) |
| Simvastatin | 19.13 ± 8.86 | 17.29 ± 8.25 | 4.06 ± 2.61 | 3.7 ± 0.95 | 5.44 ± 4.71 | 2.45 ± 0.37 |
| 16 mg - batch 1 | (3.68; 31.00) | (3.54; 30.12) | (1.19; 8.50) | (2.5; 5) | (0.76; | (2; 3) |
| (Dexcel) | N = 7 | | | | 15.51) | |
| Simvastatin | 21.60 ± 8.12 | 18.07 ± 7.39 | 2.63 ± 1.91 | 8.15 ± 6.38 | 9.62 ± 4.15 | 4.5 ± 2.55 |
| 16 mg - batch 2 | (12.63; 30.30) | (7.95; 28.90) | (0.84; 6.06) | (3; 24) | (5.52; | (2.5; 10) |
| (Dexcel) | N = 5 | | | | 16.59) | |

TABLE 11
Simvastatin Hydroxyacid - Second study

| | AUC_{(0-∞)} | AUC₍₀₋ₜ₎ | Cmax | Tmax | T½ | LagTime |
|---|---|---|---|---|---|---|
| | (ng × hour/ml) | (ng × hour/ml) | (ng/ml) | (hours) | (hr.) | (hours) |
| Simvastatin | 30.22 ± 46.16 | 28.07 ± 43.87 | 2.92 ± 4.31 | 4.45 ± 0.83 | 7.46 ± 4.87 | 2.60 ± 0.39 |
| 16 mg - batch 1 | (6.14; | (5.58; | (0.67; 15.00) | (3; 6) | (2.99; | (2; 3.5) |
| (Dexcel) | 151.74) | 150.97) | | | 16.50) | |
| | N = 9 | | | | | |
| Simvastatin | 19.74 ± 8.99 | 27.35 ± 39.35 | 1.88 ± 1.75 | 11.55 ± 9.85 | 10.67 ± 4.27 | 6.5 ± 6.64 |
| 16 mg - batch 2 | (5.98; 32.21) | (5.28; 137.52) | (0.38; 6.37) | (4.5; 36) | (3.62; | (2.5; 24) |
| (Dexcel) | N = 7 | | | | 14.20) | |

TABLE 12
Simvastatin - Comparison between the two tests from the second
study vs. the reference from the first study

| | AUC_{(0-∞)} | AUC₍₀₋ₜ₎ | Cmax | Tmax |
|---|---|---|---|---|
| | (ng × hour/ml) | (ng × hour/ml) | (ng/ml) | (hours) |
| Ratio* | 0.40 | 0.47 | 0.36 | |
| 16 mg - batch 1 vs. Ref. | (0.21; 0.74) | (0.31; 0.71) | (0.21; 0.62) | |
| (90% ANOVA C.I.) | | | | |
| Ratio* | 0.44 | 0.52 | 0.23 | |
| 16 mg - batch 2 vs. Ref. | (0.23; 0.86) | (0.31; 0.86) | (0.12; 0.44) | |
| 90% ANOVA C.I.) | | | | |
| Difference between 16 mg - | | | | 1.95 ± 1.09 |
| batch 1 vs. Ref. | | | | (0.5; 4) |
| Difference between 16 mg - | | | | 6.4 ± 6.62 |
| batch 2 vs. Ref | | | | (1.5; 23) |

TABLE 13
Simvastatin Hydroxyacid - Comparison between the two tests from the second
study vs. the reference from the first study

| | AUC_{(0-∞)} | AUC₍₀₋ₜ₎ | Cmax | Tmax |
|---|---|---|---|---|
| | (ng × hour/ml) | (ng × hour/ml) | (ng/ml) | (hours) |
| Ratio* | 1.09 | 1.13 | 0.84 | |
| 16 mg - batch 1 vs. Ref. | (0.50; 2.38) | (0.53; 2.41) | (0.44; 1.63) | |
| (90% ANOVA C.I.) | | | | |
| Ratio* | 0.98 | 1.20 | 0.66 | |
| 16 mg - batch 2 vs. Ref. | (0.52; 1.86) | (0.58; 2.49) | (0.41; 1.05) | |
| (90% ANOVA C.I.) | | | | |
| Difference between 16 mg - | | | | −0.95 ± 3.78 |
| formulation 1 vs. | | | | (−11.5; 1.5) |
| Ref. | | | | |
| Difference between 16 mg - | | | | 6.15 ± 7.17 |
| formulation 2 vs. | | | | (0; 20) |
| Ref. | | | | |

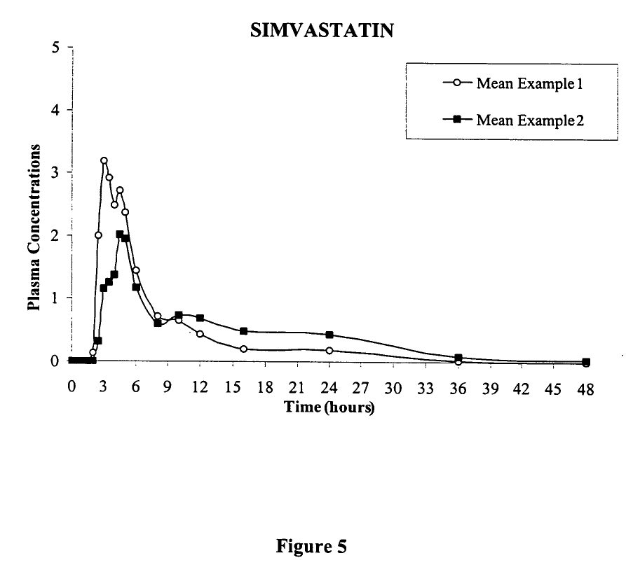

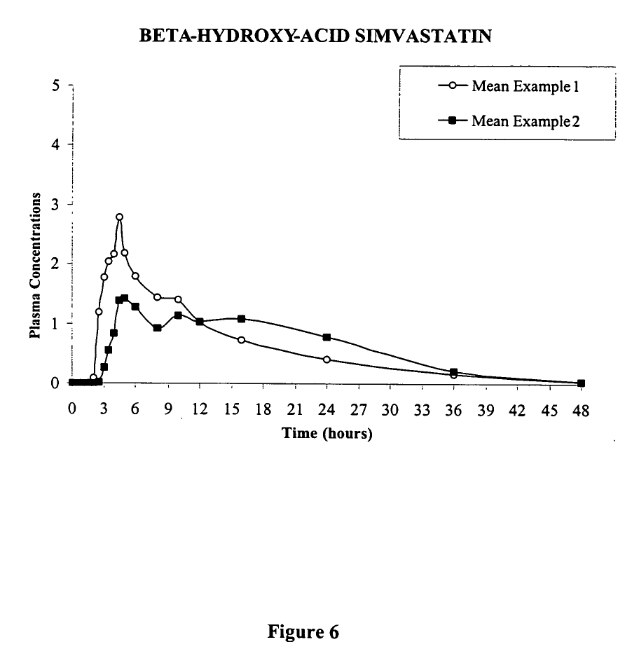

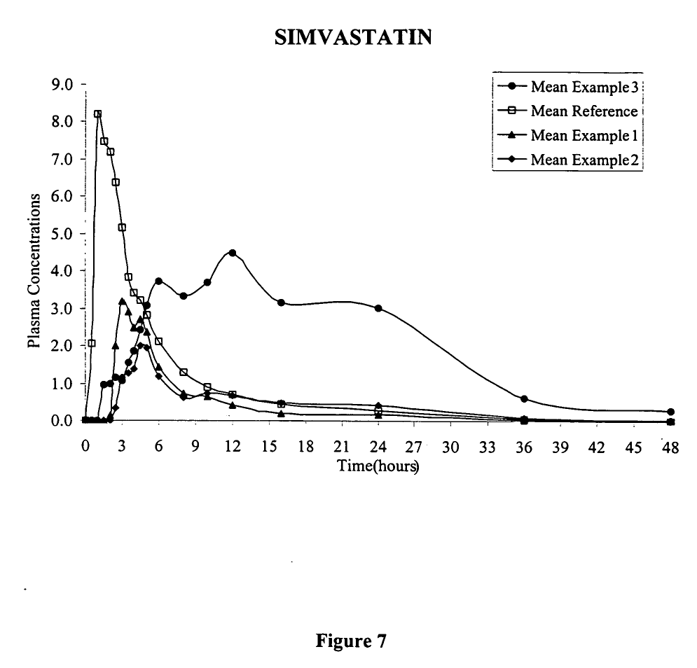

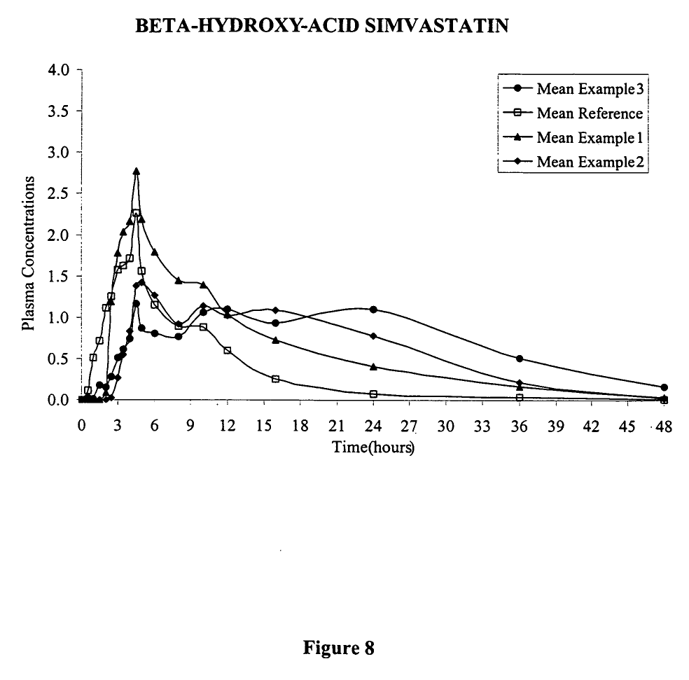

FIG. 5 shows the mean plasma simvastatin levels for two batches of 16 mg formulations, while FIG. 6 shows the mean plasma beta-hydroxy-acid simvastatin levels for the same two batches, over a 48 hour period after administration of the inventive formulation. FIG. 7 (mean plasma simvastatin levels) and FIG. 8 (mean plasma beta-hydroxy-acid simvastatin levels) show the comparison between all the inventive formulations, as compared to the reference product.

As can be seen, the formulations according to the present invention provide a longer plateau level, as compared to the reference preparation. The overall bioavailability is higher. When a dose which is 2.5 times lower (Simvastatin 16 mg) than that of the reference product (Simvastatin 40 mg) was administered, the area under the plasma concentration versus time curve up to the last measured concentration (AUC) of the active beta-hydroxy-acid metabolite was higher than that of the reference product, hence the production of the active beta-hydroxy-acid metabolite is shown to be more efficient for the formulation of the present invention as compared with that of the reference product.

Furthermore, the peak dose concentration (Cmax) for the active beta-hydroxy-acid metabolite from the present invention (16 mg formulation—batch 2) was shown to be relatively lower than that of the reference product. Time to Cmax (Tmax) was greater for the active beta-hydroxy-acid metabolite from the formulation of the present invention, as compared to the reference product and also in comparison to Tmax for the statin from which the metabolite is derived.

The results show that the formulations of the present invention can achieve a suitable level of bioavailability (and hence a suitable therapeutic level) with a lower administered dosage (in this Example, about 2.5 times lower) than with formulations known to the art. The administration of a lower dosage has the advantages of reduced side-effects, such as reduced liver transaminase; reduced incidences of rhabdomyolysis, muscle pain, and CPK; and reduced gastrointestinal effects, including reduced nausea, dyspepsia, flatulence, and/or constipation.

Bioavailability Study of Pitavastatin Calcium Salt

The Pharmacokinetic profiles of two TCDS formulations containing 4 mg and 8 mg pitavastatin calcium were compared to a comparative conventional immediate release formulation. The results show that the formulations according to the present invention can achieve a suitable level of peak concentration (Cmax) for both 4 mg and 8 mg based formulations (Cmax was found to be in the range of 70-200 ng/ml and 100-400 ng/ml for 4 mg and 8 mg pitavastatin calcium salt based formulation respectively). Likewise, TCDS formulations resulted in even levels of blood concentrations maintained for a duration of at least up to 18-24 hours for both 4 mg and 8 mg of pitavastatin calcium salt.

Part III—Efficacy Study for Simvastatin

The formulations of the present invention have increased efficacy and are capable of providing at least similar, if not greater, pharmaceutical effects with the active ingredient with a significantly decreased dosage amount as compared to other orally administered formulations that are known in the art. Without wishing to be limited by a single hypothesis, it is also possible that lower side effects may be observed with the formulation of the present invention, again as compared to other orally administered dosage forms that are known in the art.

The term “decreased dosage amount”, as used herein, refers to a dosage of a statin, which is lower than the dosage used in a corresponding conventional immediate release formulation of the statin. Optionally, such a decreased dosage amount of the active ingredient, preferably simvastatin, comprises most preferably about 50% of the regular dosage amount, optionally up to about 30% or 40% of the regular dosage amount. One non-limiting example of a conventional or “regular” dosage amount is that administered with the currently available reference product, which as noted above is the Zocor (immediate release) product of Merck. Any other immediate release product could also be considered to be a “regular” product that is known in the art. The dosage amount during a 24 hour period is also determined by the dosage frequency; preferably, the formulation of the present invention is not administered more frequently than the “regular” orally administered formulations; more preferably, the formulation of the present invention is administered once daily, optionally in the evening.

A clinical study was performed to study the efficacy of the formulations of the present invention. This study compared the efficacy of a tablet according to the present invention, with the same formulation as for the Part II studies above but containing 10 mg of simvastatin, with the Zocor reference product (also as used in the Part II studies above) which contains 20 mg of simvastatin (this is the regular dosage level of simvastatin). The clinical study was conducted with hypercholesterolemia patients, although it should be noted that this category of patients was chosen for the purpose of the study only and should not limit the indications of the present formulations in any way.

The primary end point criteria of the study demonstrated equivalent or superior mean percent reductions from baseline (i.e. before treatment) in LDL-C (LDL (low density lipoprotein) concentrations in the blood) observed in patients taking the tablet according to the present invention, as compared to the reference product (Immediate release).

Both sets of patients took one tablet per day (present invention or reference) in the evening. Each set included 80 patients having elevated cholesterol levels. The patients either not have been previously treated with a statin, or have undergone a 6 week washout period (during which no statin is given) before the study began. The study was a double-blind, randomized and multicenter.

The clinical study showed that the tablet of the present invention (with the lower dosage amount of 10 mg per tablet) is at least as clinically effective as the immediate release reference product (with 20 mg per tablet), thereby providing at least similar clinical efficacy but with a significantly lower dose.

The formulation of the present invention therefore provides a delayed onset, rapid burst release formulation for delivery of statins preferentially to the colon or small intestine, which provides fewer fluctuations in the levels of drug and/or its active form in the bloodstream, with a substantially sustained plateau. The bioavailability is shown to be higher than that of a known reference product, with the plateau maintained over a longer period, resulting in fewer side effects associated with sharp peaks and troughs, which should therefore result in greater patient compliance and comfort.

The formulation of the present invention preferably includes a burst-controlling agent, such that release occurs rapidly, within a period of less than 8 hours following oral administration, with selective absorption of the active agent in the lower parts of the small intestine or in the colon.

It is appreciated that certain features of the invention, which are, for clarity, described in the context of separate embodiments, can also be provided in combination in a single embodiment. Conversely, various features of the invention, which are, for brevity, described in the context of a single embodiment, can also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A delayed burst release oral formulation for localized release of a statin or a pharmaceutically acceptable salt, ester or an active form thereof in the gastrointestinal tract of a subject, comprising:
(a) a core comprising at least one statin, and at least one burst controlling agent, wherein the burst controlling agent is a water insoluble polymer; and
(b) an outer coating over the core, the outer coating comprising a water insoluble hydrophobic carrier and a water insoluble hydrophilic particulate matter, the water insoluble hydrophilic particulate matter allowing entry of liquid into said core.

2. The delayed burst release oral formulation for localized release of a statin in the gastrointestinal tract of a subject, according to claim 1, wherein the in vivo blood plasma concentration of said statin, a pharmaceutically acceptable salt or ester thereof or an active form thereof in the subject is substantially zero for at least about 1.5 hours after oral administration.

3. The delayed burst release oral formulation for localized release of a statin in the gastrointestinal tract of a subject, according to claim 2, wherein the in vivo blood plasma concentration of said statin, a pharmaceutically acceptable salt or ester thereof or an active form thereof in the subject is substantially zero for at least about 2 hours after oral administration.

4. The delayed burst release oral formulation for localized release of a statin in the gastrointestinal tract of a subject, according to claim 3, wherein the in vivo blood plasma concentration of said statin, a pharmaceutically acceptable salt or ester thereof or an active form thereof in the subject is substantially zero for at least about 3 hours after oral administration.

5. The delayed burst release oral formulation according to claim 1 that provides an enhanced bioavailability of a statin, a pharmaceutically acceptable salt or ester thereof, or an active form thereof to the circulation of a subject, compared to a substantially similar dose of an immediate release formulation of said statin.

6. The delayed burst release oral formulation according to claim 1 that provides enhanced bioavailability of a statin, a pharmaceutically acceptable salt or ester thereof, or an active form thereof in a subject, as measured by the AUC compared to a substantially similar dose of an immediate release formulation of said statin.

7. The delayed burst release oral formulation for localized release of a statin in the gastrointestinal tract of a subject according to claim 1, wherein said statin is present in a decreased dosage amount of up to about 60% as compared to an immediate release formulation of said statin, while providing a substantially similar bioavailability to said immediate release formulation.

8. The delayed burst release oral formulation for localized release of a statin in the gastrointestinal tract of a subject, according to claim 1, wherein the formulation releases substantially no statin in vitro for at least about 1 hour.

9. The delayed burst release oral formulation according to claim 1, wherein the formulation releases substantially no statin in vitro for at least about 1.5 hours.

10. The delayed burst release oral formulation according to claim 1, wherein the formulation releases substantially no statin in vitro for at least about 2 hours.

11. The delayed burst release oral formulation for localized release of a statin in the gastrointestinal tract of a subject according to claim 1, wherein at least about 70% of the statin is released in vitro about 1 hour after the delayed burst release occurs.

12. The delayed burst release oral formulation for localized release of a statin in the gastrointestinal tract of a subject according to claim 1, wherein said formulation provides a therapeutically effective amount of said statin, a pharmaceutically acceptable salt or ester thereof or an active form thereof in the subject for at least about 12 hours after the burst release occurs.

13. The formulation according to claim 1, wherein said water insoluble hydrophilic particulate matter forms channels in said outer coating upon contact with a liquid, whereby said channels absorb said liquid and cause said at least one burst controlling agent to burst said coating, thereby providing delayed burst release of said statin.

14. The formulation according to claim 1, wherein said statin is selected from the group consisting of simvastatin, lovastatin, mevastatin, pravastatin, fluvastatin, atorvastatin, pitavastatin and rivastatin.

15. The formulation according to claim 14, wherein said statin is simvastatin.

16. The formulation according to claim 1, wherein said active form is the hydroxy acid form

17. The formulation according to claim 1, wherein said formulation preferentially releases said statin in the intestine of the subject.

18. The formulation according to claim 1, wherein said formulation preferentially releases statin in the lower gastrointestinal tract of the subject.

19. The formulation according to claim 1, wherein said formulation preferentially releases statin in the colon of the subject.

20. The formulation according to claim 1, wherein said core is in a form selected from the group consisting of a tablet, a pellet, microparticles, an agglomerate, a pill and a capsule.

21. The formulation according to claim 1, wherein said water insoluble polymer is selected from the group consisting of a cross-linked polysaccharide, a water insoluble starch, microcrystalline cellulose, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, and cross-linked polyacrylic acid.

22. The formulation according to claim 21, wherein said cross-linked polysaccharide is selected from the group consisting of insoluble metal salts or cross-linked derivatives of alginate, pectin, xanthan gum, guar gum, tragacanth gum, locust bean gum, and carrageenan.

23. The formulation according to claim 21, wherein said modified cellulose is selected from the group consisting of cross-linked derivatives of hydroxypropylcellulose, hydroxypropylmethylcellulose, hydroxyethyl cellulose, methylcellulose, carboxymethylcellulose, and metal salts of carboxymethylcellulose.

24. The formulation according to claim 21, wherein said water insoluble polymer is calcium pectinate, microcrystalline cellulose or a combination thereof.

25. The formulation according to claim 1, wherein the core further comprises at least one disintegrant.

26. The formulation according to claim 25, wherein said disintegrant is selected from the group consisting of cross-linked polyvinylpyrrolidinone, sodium starch glycolate, cross-linked sodium carboxymethylcellulose, pregelatinized starch, microcrystalline starch, water insoluble starch, calcium carboxymethylcellulose, magnesium aluminum silicate, and combinations thereof.

27. The formulation according to claim 1, wherein said water-insoluble hydrophobic carrier is selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, said polymer corresponding to USP/NF “Ammonio Methacrylate Copolymer Type A”; an ethylmethacrylate/chlorotrimethyl ammonium ethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”; a dimethylaminoethylmethacrylate/methylmethacrylate and butylmethacrylate copolymer; a copolymer based on neutral methacrylic acid esters and dimethylaminoethyl methacrylate esters wherein the polymer is cationic in the presence of acids; an ethylacrylate and methylacrylate/ethylmethacrylate; and a methyl methylacrylate copolymer, the copolymer being a neutral copolymer based on neutral methacrylic acid and acrylic acid esters; ethylcellulose; shellac; and waxes.

28. The formulation according to claim 27, wherein said water-insoluble hydrophobic carrier is ethylcellulose.

29. The formulation according to claim 1, wherein said water insoluble hydrophilic particular matter is selected from the group consisting of a water insoluble polysaccharide, a water insoluble cross-linked polysaccharide, a water insoluble polysaccharide metal salt including calcium pectinate, a water insoluble cross-linked protein, a water insoluble cross-linked peptide, water insoluble cross-linked gelatin, water insoluble cross-linked hydrolyzed gelatin, water insoluble cross-linked collagen, a water insoluble cross linked polyacrylic acid, a water insoluble cross-linked cellulose derivative, water insoluble cross-linked polyvinyl pyrrolidone, microcrystalline cellulose, insoluble starch, microcrystalline starch and any combination thereof.

30. The formulation according to claim 29, wherein said water insoluble hydrophilic particular matter is microcrystalline cellulose.

31. The formulation according to claim 1, wherein said core further comprises at least one of a binder, an absorption enhancer, a hardness enhancing agent, a buffering agent, a filler, a flow regulating agent, a lubricant, a chelator, a synergistic agent, an antioxidant, a stabilizer and a preservative.

32. The formulation according to claim 1, wherein said outer coating further comprises at least one of a wetting agent, a suspending agent, a dispersing agent, a stiffening agent and a plasticizer.

33. The formulation according to claim 1, further comprising an enteric coating disposed over said outer coating.

34. The formulation according to claim 33, wherein said enteric coating is selected from the group consisting of hydroxypropylmethyl cellulose phthalate, polyvinyl acetate phthalate, cellulose acetate phthalate, hydroxy propyl methyl cellulose acetate succinate, poly(methacrylic acid, methyl methacrylate) 1:1 (Eudragit L100), poly(methacrylic acid, ethyl acrylate) 1:1 (Eudragit L30D-55), alginic acid, sodium alginate.

35. The formulation according to claim 33, wherein said enteric coating comprises a methacrylic acid copolymer.

36. The formulation according to claim 33, wherein said enteric coating further comprises a plasticizer.

37. The formulation according to claim 1, wherein the statin is released in vivo at least about 3 hours after oral administration.

38. The formulation according to claim 1, wherein the statin is released in vivo after at least about 4 hours after oral administration.

39. The formulation according to claim 1, wherein said statin is present in a decreased dosage amount of up to about 60% as compared to an immediate release formulation of said statin, while providing a substantially similar therapeutic effect to said immediate release formulation.

40. The formulation according to claim 1, wherein said formulation provides a therapeutically effective amount of said statin, a pharmaceutically acceptable salt or ester thereof or an active form thereof in the subject for at least about 12 hours after the burst release occurs.

41. The formulation according to claim 1, wherein said formulation provides a therapeutically effective amount of said statin, a pharmaceutically acceptable salt or ester thereof or an active form thereof in the subject for at least about 24 hours after the burst release occurs.

42. The formulation according to claim 1, wherein said formulation releases said statin in the gastrointestinal tract, and provides clinically effective amounts of a hydroxy acid metabolite of said statin into the circulation of the subject.

43. A method for providing a therapeutically effective amount of a statin, a pharmaceutically acceptable salt or ester thereof or an active form thereof to a subject, comprising orally administering to the subject a delayed burst release formulation comprising:
(a) a core comprising at least one statin or a pharmaceutically acceptable salt or ester thereof, and at least one burst controlling agent, wherein the burst controlling agent is a water insoluble polymer; and
(b) an outer coating over the core, the outer coating comprising a water insoluble hydrophobic carrier and a water insoluble hydrophilic particulate matter, the water insoluble hydrophilic particulate matter allowing entry of liquid into said core.

44. A method for providing enhanced bioavailability of a statin, a pharmaceutically acceptable salt or ester thereof or an active form thereof to the circulation of a subject, comprising orally administering to the subject a delayed burst release formulation comprising:
(a) a core comprising at least one statin or a pharmaceutically acceptable salt or ester thereof, and at least one burst controlling agent, wherein the burst controlling agent is a water insoluble polymer; and
(b) an outer coating over the core, the outer coating comprising a water insoluble hydrophobic carrier and a water insoluble hydrophilic particulate matter, the water insoluble hydrophilic particulate matter allowing entry of liquid into said core.

45. A method of providing fast release of a statin a pharmaceutically acceptable salt or ester thereof or an active form thereof in the gastrointestinal tract of a subject, comprising orally administering to the subject a delayed burst release formulation comprising:
(a) a core comprising at least one statin or a pharmaceutically acceptable salt or ester thereof, and at least one burst controlling agent, wherein the burst controlling agent is a water insoluble polymer; and
(b) an outer coating over the core, the outer coating comprising a water insoluble hydrophobic carrier and a water insoluble hydrophilic particulate matter, the water insoluble hydrophilic particulate matter allowing entry of liquid into said core.

46. The method for providing a therapeutically effective amount of a statin, a pharmaceutically acceptable salt or ester thereof or an active form thereof to a subject, according to claim 45, wherein said formulation provides a therapeutically effective amount of said statin, a pharmaceutically acceptable salt or ester thereof or an active form thereof in the subject for at least about 12 hours after the burst release occurs.

47. The method according to claim 45, wherein said water insoluble hydrophilic particulate matter forms channels in said outer coating upon contact with a liquid, whereby said channels absorb said liquid and cause said at least one burst controlling agent to burst said coating, thereby providing delayed burst release of said statin.

48. The method according to claim 47, wherein the statin is selected from the group consisting of simvastatin, lovastatin, mevastatin, pravastatin, fluvastatin, atorvastatin, pitavastatin and rivastatin.

49. The method according to claim 48, wherein the statin is simvastatin.

50. The method according to claim 47, wherein said formulation preferentially releases said statin in the intestine of the subject.

51. The method according to claim 47, wherein said formulation preferentially release statin in the lower gastrointestinal tract of the subject.

52. The method according to claim 47, wherein said formulation preferentially release statin in the colon of the subject.

53. The method according to claim 47, wherein said core is in a form selected from the group consisting of a tablet, a pellet, microparticles, an agglomerate, a pill and a capsule.

54. The method according to claim 47, wherein said water insoluble polymer is selected from the group consisting of a cross-linked polysaccharide, a water insoluble starch, microcrystalline cellulose, a water insoluble cross-linked peptide, a water insoluble cross-linked protein, a water insoluble cross-linked gelatin, a water insoluble cross-linked hydrolyzed gelatin, a water insoluble cross-linked collagen, a modified cellulose, and cross-linked polyacrylic acid.

55. The method according to claim 54, wherein said cross-linked polysaccharide is selected from the group consisting of insoluble metal salts or cross-linked derivatives of alginate, pectin, xantham gum, guar gum, tragacanth gum, locust bean gum, and carrageenan.

56. The method according to claim 54, wherein said modified cellulose is selected from the group consisting of cross-linked derivatives of hydroxypropylcellulose, hydroxypropylmethylcellulose, hydroxyethyl cellulose, methylcellulose, carboxymethylcellulose, and metal salts of carboxymethylcellulose.

57. The method according to claim 54, wherein said water insoluble polymer is calcium pectinate, microcrystalline cellulose or a combination thereof.

58. The method according to claim 47, wherein the core further comprises at least one disintegrant.

59. The method according to claim 58, wherein said disintegrant is selected from the group consisting of cross-linked polyvinylpyrrolidinone, sodium starch glycolate, cross-linked sodium carboxymethylcellulose, pregelatinized starch, microcrystalline starch, water insoluble starch, calcium carboxymethylcellulose, magnesium aluminum silicate, and combinations thereof.

60. The method according to claim 47, wherein said water-insoluble hydrophobic carrier is selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, said polymer corresponding to USP/NF “Ammonio Methacrylate Copolymer Type A”; an ethylmethacrylate/chlorotrimethyl ammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”; a dimethylaminoethylmethacrylate/methylmethacrylate and butylmethacrylate copolymer; a copolymer based on neutral methacrylic acid esters and dimethylaminoethyl methacrylate esters wherein the polymer is cationic in the presence of acids; an ethylacrylate and methylacrylate/ethylmethacrylate; and a methyl methylacrylate copolymer, the copolymer being a neutral copolymer based on neutral methacrylic acid and acrylic acid esters; ethylcellulose; shellac; and waxes.

61. The method according to claim 60, wherein said water-insoluble hydrophobic carrier is ethylcellulose.

62. The method according to claim 47, wherein said water insoluble hydrophilic particular matter is selected from the group consisting of a water insoluble polysaccharide, a water insoluble cross-linked polysaccharide, a water insoluble polysaccharide metal salt including calcium pectinate, a water insoluble cross-linked protein, a water insoluble cross-linked peptide, water insoluble cross-linked gelatin, water insoluble cross-linked hydrolyzed gelatin, water insoluble cross-linked collagen, a water insoluble cross linked polyacrylic acid, a water insoluble cross-linked cellulose derivatives, water insoluble cross-linked polyvinyl pyrrolidone, microcrystalline cellulose, insoluble starch, microcrystalline starch and a combination thereof.

63. The method according to claim 62, wherein said water insoluble hydrophilic particular matter is microcrystalline cellulose.

64. The method according to claim 47, wherein said core further comprises at least one of a binder, an absorption enhancer, a hardness enhancing agent, a buffering agent, a filler, a flow regulating agent, a lubricant, a chelator, a synergistic agent, an antioxidant, a stabilizer and a preservative.

65. The method according to claim 47, wherein said outer coating further comprises at least one of a wetting agent, a suspending agent, a dispersing agent, a stiffening agent and a plasticizer.

66. The method according to claim 47, further comprising an enteric coating disposed over said outer coating.

67. The method according to claim 66, wherein said enteric coating is selected from the group consisting of hydroxypropylmethyl cellulose phthalate, polyvinyl acetate phthalate, cellulose acetate phthalate, hydroxy propyl methyl cellulose acetate succinate, poly(methacrylic acid, methyl methacrylate) 1:1 (Eudragit L100), poly(methacrylic acid, ethyl acrylate)1:1 (Eudragit L30D-55), alginic acid, sodium alginate.

68. The method according to claim 66, wherein said enteric coating comprises a methacrylic acid copolymer.

69. The method according to claim 66, wherein said enteric coating further comprises a plasticizer.

70. The method according to claim 47, wherein the statin is released in vivo at least about 3 hours after oral administration.

71. The method according to claim 47, wherein the statin is released in vivo at least about 4 hours after oral administration.

72. The method according to claim 47, wherein said statin is present in a decreased dosage amount of up to about 60% as compared to an immediate release formulation of said statin, while providing a substantially similar therapeutic effect to said immediate release formulation.

73. The method according to claim 47, wherein the in vivo blood plasma concentration of said statin, a pharmaceutically acceptable salt or ester thereof or an active form thereof in the subject is substantially zero for at least about 1.5 hours after oral administration.

74. The method according to claim 47, wherein the in vivo blood plasma concentration of said statin, a pharmaceutically acceptable salt or ester thereof or an active form thereof in the subject is substantially zero for at least about two hours after oral administration.

75. The method according to claim 47, wherein said formulation provides a therapeutically effective amount of said statin, a pharmaceutically acceptable salt or ester thereof or an active form thereof in the subject for at least about 12 hours after the burst release occurs.

76. The method according to claim 47, wherein said formulation provides a therapeutically effective amount of said statin, a pharmaceutically acceptable salt or ester thereof or an active form thereof in the subject for at least about 24 hours after the burst release occurs.

77. A delayed burst release oral formulation according to claim 1, wherein said statin is present in a decreased dosage amount of up to about 50% as compared to an immediate release formulation of said statin, while providing a substantially similar lowering of LDL as said immediate release formulation.
